(19) United States
(12) Patent Application Publication
(10) Pub. No.: US 2012/0095048 A1
(43) Pub. Date: Apr. 19, 2012

(54) ALLOSTERIC BINDING COMPOUNDS

(75) Inventors: Henrik Amtoft Neubauer, Ega (DK); Ove Kjaer Wiborg, Aarhus N (DK); Henrik Helligsø Jensen, Aarhus N (DK)

(73) Assignees: REGION MIDT JYLLAND, Viborg (DK); AARHUS UNIVERSITET, Aarhus C (DK)

(21) Appl. No.: 13/202,495

(22) PCT Filed: Feb. 19, 2010
(86) PCT No.: PCT/DK2010/050044

§ 371 Date: Jan. 3, 2012

(30) Foreign Application Priority Data
Feb. 20, 2009 (DK) PA 2009 00240

(51) Int. Cl. A61K 31/4525; A61K 31/235; C07C 69/767; C07C 233/65; A61K 31/166; C07C 321/28; A61K 31/10; C07C 323/19; C07C 323/32; A61K 31/135; C07C 43/20; A61K 31/085; C07C 323/62; A61K 31/192; C07D 209/08; A61K 31/404; C07D 249/18; A61K 31/4192; A61K 31/277; A61K 31/343; A61K 31/138; A61K 31/15; A61K 31/137; A61K 31/381; A61K 31/4406; A61P 25/00; A61P 25/24; A61P 25/22; A61P 25/04; C07C 69/76

(52) U.S. Cl. 514/321; 560/100; 514/510; 564/180; 514/617; 568/58; 514/712; 568/55; 514/713; 564/341; 514/649; 568/632; 514/717; 560/10; 562/427; 514/569; 548/510; 514/415; 548/261; 514/359; 548/491; 558/396; 514/469; 514/651; 514/657; 514/640; 514/653; 514/438; 514/357

(57) ABSTRACT

The present invention relates to allosteric binding compounds of formula (I), especially for the treatment of CNS disorders, together with pharmaceutical compositions and methods of treatment including these compounds.

### FIELD OF INVENTION

The present invention relates to allosteric binding compounds of formula (I), especially for the treatment of CNS disorders, together with pharmaceutical compositions and methods of treatment including these compounds.

### BACKGROUND OF INVENTION

Serotonin is localized in the central and peripheral nervous systems and is known to affect many types of conditions including CNS disorders, psychiatric disorders, motor activity, feeding behavior, sexual activity, and neuroendocrine regulation among others.

Serotonergic neurotransmission is modulated by clearance of serotonin (5-hydroxytryptamine or 5-HT). The clearance of 5-HT from the synaptic cleft is maintained by the serotonin transporter (SERT). The transporter therefore affects the magnitude and duration of the signaling, and thus plays a key role in the spatio temporal fine tuning of serotonergic neurotransmission

The serotonin transporter (SERT), which belongs to a family of sodium/chloride-dependent transporters, is the major pharmacological target in the treatment of several clinical disorders, including depression and anxiety. Activation of a low affinity allosteric site on SERT modulates the ligand affinity at the high affinity binding site. Serotonin (5-HT), as well as some SERT inhibitors possesses affinity for both sites.

SERT is a well established molecular target of drugs of abuse (cocaine and amphetamines), as well as a number of high-affinity antidepressants. Multiple classes of antidepressants including tricyclic antidepressants, 5-HT selective reuptake inhibitors and antidepressants with dual or triple actions are directed towards SERT. They enhance serotonergic neurotransmission by inhibiting 5-HT reuptake in a competitive manner with inhibitory constants in the low nanomolar range (Barker and Blakely, 1995; Owens et al., 1997; Tatsumi et al., 1997).

Dissociation of the tricyclic imipramine from platelet membranes is attenuated in the presence of 5-HT (Wennogle and Meyerson, 1982; Wennogle and Meyerson, 1985) suggesting that 5-HT acts at a site distinct from the imipramine binding site. Several high affinity SERT inhibitors (e.g. citalopram, paroxetine, sertraline, imipramine) can also act as allosteric ligands (Plenge and Mellerup, 1985; Plenge et al., 1991). The affinity-modulating or allosteric site has been shown to be present at all three monoamine transporters, which in addition to SERT also includes transporters for dopamine and norepinephrine (Plenge and Mellerup, 1997).

The interaction with the allosteric binding site is specific for SERT as supported by several findings. Strong effects on dissociation rates are only exerted by a subset of drugs tested (Plenge et al., 1991; Chen et al., 2005). The effect is stereo selective, as some enantiomers have different potencies (Plenge et al., 1991). Species differences have been reported, concerning the allosteric potency of specific drugs (Plenge et al., 1991). A species scanning mutagenesis study comparing human and chicken SERT revealed that nine residues in the C-terminal part were an important part of an allosteric mechanism that mediated the allosteric effect of e.g. escitalopram (Neubauer et al, 2006).

Serotonin-selective reuptake inhibitors (SSRIs), such as fluoxetine, have traditionally been the mainstay of treatment for clinical depression—replacing the more toxic tricyclic antidepressants (TCAs). SSRIs have a more favourable adverse reaction profile in comparison to the TCAs and are much easier to tolerate. SSRIs exert their therapeutic effect by blocking the reuptake of serotonin into the presynaptic nerve terminal, thus increasing the synaptic concentration of serotonin. It is also believed that SSRIs increase the efficacy of the serotonin (5-HT) neurons by desensitizing 5-HT autoreceptors located on the presynaptic 5-HT nerve terminals. The ability of the 5-HT autoreceptors to inhibit the release of 5-HT decreases after long-term treatment with SSRIs, with the net effect being that a greater amount of 5-HT is released per impulse.

In WO 2007/076875 there are described compounds acting on the serotonin transporter, these compounds differ from the compounds of the present invention of formula (I), and have been found to be less active than compounds of the present invention.

Depression is a common, life-disrupting, potentially lethal illness that can affect both sexes and all ages. Untreated major depression remains a serious public health problem and its incidences are staggering. Its peak onset is in the early adult years.

Suicide occurs in as many as 15% of patients with depression, especially those with recurrent episodes and hospitalizations. Therefore it becomes evident that treatment of depression is a matter of prime importance. Depression has no single cause; often, it results from a combination of factors. Whatever its cause, depression is not just a state of mind; it is related to physical changes in the brain, and connected to an imbalance of neurotransmitters. Among the most important neurotransmitters related with depression are serotonin (5-HT), norepinephrine (NE), and dopamine (DA). Serotonin plays a very important role in the mood disorders, especially in anxiety and depression, aggression and impulsivity. Regulation of the mood disorders is possible either by agonistic or antagonistic action on a certain type of the serotonin receptors. SSRIs are primarily used for the treatment of depression, but are also used in the treatment of diseases like panic disorder, anxiety, obsessive-compulsive disorder (OCD), generalized anxiety disorder (GAD), social phobia, bulimia nervosa, anorexia nervosa, post-traumatic stress disorder (PTSD), and neuropathic pain.

Unfortunately, there is a delay in the effect of SSRIs ranging from three to four weeks, or even longer, from the onset of treatment. Symptoms may sometimes even worsen during the first weeks of treatment. In order to treat the patient during the delayed effect of SSRI, additional antidepressants may be used to augment the SSRI therapy by co-administration of compounds stabilizing the mood of the patient—such as for example lithium carbonate ortriiodothyronin or by the use of electroshock.

There is thus a need for a treatment for CNS disorders, which does not include a delayed effect, or a treatment which utilises substances that at least gives a faster onset of action compared to known active substances. Furthermore, or alternatively, there is a need for substances that may be administered in combination with existing active substances, such as e.g. anti-depressant drugs, in order to decrease the delay in the effect of the active substance, e.g. an anti-depressant drug.

### SUMMARY OF INVENTION

The present invention relates to compounds of formula (I)

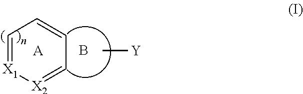

or a pharmaceutical acceptable salt, solvate or prodrug thereof;

wherein

Y is selected from

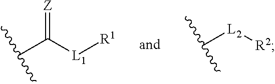

A is a 5- or 6-membered aryl or heteroaryl ring;

n is 0 or 1;

B is a 4-, 5-, or 6-membered cycloalkyl, aryl, heterocyclyl or heteroaryl ring, which ring together with A forms an annulated ring system;

X₁ and X₂ are each independently an atom selected from the group consisting of Carbon, Nitrogen, Oxygen, and Sulphur, wherein at least one of X₁ and X₂ is Carbon;

Z is an atom selected from the group consisting of Oxygen and Sulphur, with the proviso that when Z is Sulphur, then L₁ is —NH— or —NR³—;

L₁ is a linker selected from the group consisting of —O—, —NH—, —NR³—, and —C—;

L₂ is a linker selected from the group consisting of —O—, —S—, —NH—, and —NR⁴—;

R¹ is selected from the group consisting of C₁₋₈ alkyl, C₁₋₈ alkoxy, C₂₋₈ alkenyl, C₂₋₈ alkynyl, C₃₋₁₀ cycloalkyl, aryl, heterocyclyl, heteroaryl, —NH—C₁₋₆ alkyl, C₃₋₁₀ cycloalkyl-C₁₋₆ alkyl, aryl-C₁₋₆ alkyl, heterocyclyl-C₁₋₆ alkyl, and heteroaryl-C₁₋₆ alkyl;

R² is selected from the group consisting of C₁₋₈ alkyl, C₂₋₈ alkenyl, alkynyl, C₃₋₁₀ cycloalkyl, aryl, heterocyclyl, heteroaryl, C₃₋₁₀ cycloalkyl-C₁₋₆ alkyl, aryl-C₁₋₆ alkyl, heterocyclyl-C₁₋₆ alkyl, and heteroaryl-C₁₋₆ alkyl;

where any alkyl, alkenyl, alkynyl, cycloalkyl, aryl, heterocyclyl, and heteroaryl of R¹ and R² each independently optionally is substituted with one or more substituents selected from the group consisting of halogen, —OH, —SH, —NO₂, —CN, —NH₂, —N₃, C₁₋₆ alkyl, C₁₋₆ alkoxy, —COOH, —C(O)O—(C₁₋₆ alkyl), —C(O)—NH₂, —C(O)—NH(C₁₋₄ alkyl), —NH(C₁₋₆ alkyl), —N(C₁₋₄ alkyl)(C₁₋₄ alkyl), —NHC(O)—(C₁₋₆ alkyl), —S—(C₁₋₄ alkyl), —S(O)—(C₁₋₄ alkyl), —SO₂—(C₁₋₄ alkyl), —CCl₃, —CF₃, and —CH₂CF₃;

R³ is selected from the group consisting of C₁₋₆ alkyl, C₂₋₆ alkenyl, and C₂₋₆ alkynyl;

R⁴ is selected from the group consisting of C₁₋₆ alkyl, C₂₋₆ alkenyl, and C₂₋₆ alkynyl; and

where ring A and ring B of formula (I) each independently optionally is substituted with one or more substituents selected from the group consisting of halogen, —OH, —SH, —NO₂, —CN, —NH₂, —N₃, C₁₋₄ alkyl, C₁₋₄ alkoxy, C₂₋₄ alkenyl, C₂₋₄ alkynyl, —COOH, —C(O)—NH₂, —NH(C₁₋₄ alkyl), —S—(C₁₋₄ alkyl), —CF₃, and —CH₂CF₃.

The invention furthermore relates to these compounds of formula (I) for use as a medicament, preferably for treatment of CNS disorders, such as e.g. a CNS disorder selected from the group consisting of depression, panic disorder, anxiety, obsessive-compulsive disorder (OCD), generalized anxiety disorder (GAD), social phobia, bulimia nervosa, anorexia nervosa, post-traumatic stress disorder (PTSD), and neuropathic pain, and for use of the compounds in a method of treating a CNS disorder in a subject.

The compounds of formula (I) according to the invention may furthermore be used in a combination treatment with one or more further active substances, preferably one or more psychiatric medications, such as e.g. an antidepressant.

### DESCRIPTION OF DRAWINGS

FIG. 1. Dissociation of [³H]-escitalopram from SERT in the presence of increasing amounts of allosteric compound. The concentrations of allosteric compound 1-naphthoic acid ethyl ester (Example 1) in the dissociation buffer range from 400-0.2 μM.

FIG. 2. The EC₅₀ value is determined as the concentration of allosteric compound which induces a 50% attenuation of the off-rate of the radioligand, compared to the off-rate of radioligand in the absence of allosteric compound. FIG. 2 shows the off-rate at a given concentration normalized to the off-rate of the radioligand in the absence of allosteric compound 1-naphthoic acid ethyl ester (Example 1).

FIG. 3. Determination of the maximum stabilization factor. The factor is defined as the estimated upper plateau of the sigmoidal response curve, and describes how many folds the off-rate of bound radioligand from SERT can be attenuated in the presence of increasing concentrations of allosteric compound in the dissociation buffer. FIG. 3 shows the curve used to determine the maximum stabilization factor for 1-naphthoic acid ethyl ester.

FIG. 4. Effect of allosteric compound on S-citalopram IC₅₀. The inhibitory effect of an allosteric compound is determined by measuring IC₅₀ values of S-citalopram in the absence and in the presence of the allosteric compound at various concentrations. FIG. 4 shows that the IC₅₀ value decreases as the concentration of allosteric compound increases, which indicates that the presence of the allosteric compound shows an add-on inhibitory effect of the uptake of serotonin.

FIGS. 5A and 5B. Effect of allosteric compound in the mouse forced swim test (FST). Drugs were dissolved in 10% β-2-hydroxy-cyclodextrine. Male BalB/c were injected intraperitoneally with 500 μl SSRI alone or in combination with allosteric compound 30 min prior to FST (n=8). Vehicle treated animals each received 500 μl 10% β-2-hydroxy-cyclodextrine. FIG. 5A shows the effect of 5 mg/(kg bodyweight) of fluoxetine alone and in combination with 15 mg/(kg bodyweight) of the allosteric compound of Example 1 (denoted ALN10 in figure). FIG. 5B depicts the effect of 5 mg/(kg bodyweight) of escitalopram alone and in combination with 15 mg/(kg bodyweight) of the allosteric compound of Example 1 (denoted ALN10 in figure).

### DETAILED DESCRIPTION OF THE INVENTION

The present invention relates to compounds of formula (I), or pharmaceutical acceptable salts, solvates or prodrugs thereof, for the treatment of CNS disorders.

The inventors have surprisingly found that compounds of formula (I), including compounds represented by subformulas thereof described herein, can be used in the treatment of CNS disorders. The present inventors have furthermore found that these compounds of formula (I) can be used in combination with psychiatric medication, such as e.g. antidepressants, to provide a synergistic effect whereby a faster onset of action of the psychiatric medication is induced. Accordingly, it is an object of the present invention to provide compounds that can be used for the treatment of CNS disorders. It is furthermore an object of the present invention to provide compounds that in combination with psychiatric medication, such as e.g. SSRI, SNRI or other antidepressants, gives a synergistic effect, thereby inducing a faster onset of action of the psychiatric medication. The compounds of formula (I) have been found to have an improved activity profile compared to compounds previously reported in WO 2007/076875.

Furthermore the present inventors have surprisingly found that the compounds of formula (I), including compounds represented by subformulas thereof described herein, can be used in combination with psychiatric medication, such as e.g. antidepressants, to provide an increased efficacy. Accordingly, it is an object of the present invention to provide compounds that given in combination with psychiatric medication, such as e.g. SSRI, SNRI or other antidepressants, increases the efficacy of the psychiatric medication.

Accordingly, a first aspect of the present invention relates to a compound of formula (I)

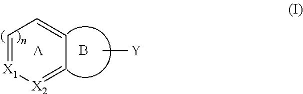

or a pharmaceutical acceptable salt, solvate or prodrug thereof;

wherein

Y is selected from

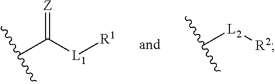

A is a 5- or 6-membered aryl or heteroaryl ring;

n is 0 or 1;

B is a 4-, 5-, or 6-membered cycloalkyl, aryl, heterocyclyl or heteroaryl ring, which ring together with A forms an annulated ring system;

X₁ and X₂ are each independently an atom selected from the group consisting of Carbon, Nitrogen, Oxygen, and Sulphur, wherein at least one of X₁ and X₂ is Carbon;

Z is an atom selected from the group consisting of Oxygen and Sulphur, with the proviso that when Z is Sulphur, then L₁ is —NH— or —NR³—;

L₁ is a linker selected from the group consisting of —O—, —NH—, —NR³—, and —C—;

L₂ is a linker selected from the group consisting of —O—, —S—, —NH—, and —NR⁴—;

R¹ is selected from the group consisting of C₁₋₈ alkyl, C₁₋₈ alkoxy, C₂₋₈ alkenyl, C₂₋₈ alkynyl, C₃₋₁₀ cycloalkyl, aryl, heterocyclyl, heteroaryl, —NH—C₁₋₆ alkyl, C₃₋₁₀ cycloalkyl-C₁₋₆ alkyl, aryl-C₁₋₆ alkyl, heterocyclyl-C₁₋₆ alkyl, and heteroaryl-C₁₋₆ alkyl;

R² is selected from the group consisting of C₁₋₈ alkyl, C₂₋₈ alkenyl, C₂₋₈ alkynyl, C₃₋₁₀ cycloalkyl, aryl, heterocyclyl, heteroaryl, C₃₋₁₀ cycloalkyl-C₁₋₆ alkyl, aryl-C₁₋₆ alkyl, heterocyclyl-C₁₋₆ alkyl, and heteroaryl-C₁₋₆ alkyl;

where any alkyl, alkenyl, alkynyl, cycloalkyl, aryl, heterocyclyl, and heteroaryl of R¹ and R² each independently optionally is substituted with one or more substituents selected from the group consisting of halogen, —OH, —SH, —NO₂, —CN, —NH₂, —N₃, C₁₋₆ alkyl, C₁₋₆ alkoxy, —COOH, —C(O)O—(C₁₋₆ alkyl), —C(O)—NH₂, —C(O)—NH(C₁₋₄ alkyl), —NH(C₁₋₆ alkyl), —N(C₁₋₄ alkyl)(C₁₋₄ alkyl), —NHC(O)—(C₁₋₆ alkyl), —S—(C₁₋₄ alkyl), —S(O)—(C₁₋₄ alkyl), —SO₂—(C₁₋₄ alkyl), —CCl₃, —CF₃, and —CH₂CF₃;

R³ is selected from the group consisting of C₁₋₆ alkyl, C₂₋₆ alkenyl, and C₂₋₆ alkynyl;

R⁴ is selected from the group consisting of C₁₋₆ alkyl, C₂₋₆ alkenyl, and C₂₋₆ alkynyl; and

where ring A and ring B of formula (I) each independently optionally is substituted with one or more substituents selected from the group consisting of halogen, —OH, —SH, —NO₂, —CN, —NH₂, —N₃, C₁₋₄ alkyl, C₁₋₄ alkoxy, C₂₋₄ alkenyl, C₂₋₄ alkynyl, —COOH, —C(O)—NH₂, —NH(C₁₋₄ alkyl), —S—(C₁₋₄ alkyl), —CF₃, and —CH₂CF₃.

A second aspect of the present invention relates to a compound of formula (I), as defined herein, for use as a medicament.

A third aspect of the present invention relates to a compound of formula (I), as defined herein, for treatment of a CNS disorder.

A fourth aspect of the present invention relates to use of a compound of formula (I), as defined herein, for the manufacture of a medicament for the treatment of a CNS disorder.

A fifth aspect of the present invention relates to a compound of formula (I), as defined herein, for treatment of a CNS disorder, wherein a compound of the present invention is administered in combination with one or more further active substance, such as e.g. one or more further psychiatric medications.

The term “alkyl”, as used herein, unless otherwise indicated, includes saturated monovalent hydrocarbon radicals having straight or branched moieties. Examples of alkyl moieties include, but are not limited to, methyl, ethyl, propyl, isopropyl, n-butyl, iso-butyl, sec-butyl, tert-butyl, and neopentyl. Alkyl is preferably C₁-C₆ alkyl, i.e. groups containing from 1 to 6 carbon atoms, and for some embodiments of the present invention, more preferably C₁-C₄ alkyl.

The term “alkenyl”, as used herein, unless otherwise indicated, includes alkyl moieties having at least one carbon-carbon double bond wherein alkyl is as defined above. Examples of alkenyl include, but are not limited to, ethenyl, propenyl, 1-butenyl, and 2-butenyl. Alkenyl is preferably C₂-C₆ alkyl, i.e. groups containing from 2 to 6 carbon atoms, and for some embodiments of the present invention, more preferably C₁-C₄ alkenyl.

The term “alkynyl”, as used herein, unless otherwise indicated, includes alkyl moieties having at least one carbon-carbon triple bond wherein alkyl is as defined above. Examples of alkynyl groups include, but are not limited to, ethynyl, 2-propynyl, 1-butynyl, and 2-butynyl.

The term “alkoxy”, as used herein, means an —O-alkyl group wherein “alkyl” is as defined above. Alkoxy furthermore refers to polyethers such as —O—(CH₂)₁₋₆—O—CH₃. Examples include, but are not limited to methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, and 3-methylpentoxy. Alkoxy is preferably C₁-C₆ alkoxy, i.e. groups containing from 1 to 6 carbon atoms, and for some embodiments of the present invention, more preferably C₁-C₄ alkoxy.

The term “cycloalkyl”, as used herein, unless otherwise indicated, includes non-aromatic saturated cyclic alkyl moieties wherein alkyl is as defined above. Cycloalkyl furthermore includes saturated carbocyclic groups consisting of two or more rings, such as spiro ring systems, fused ring systems and bridged ring systems, wherein said rings share one or two carbon atoms. Cycloalkyl also include groups that are substituted with one or more oxo moieties. Examples of cycloalkyl include, but are not limited to, cyclopropyl, cyclobutyl, cyclopentyl, cyclohexyl, cycloheptyl, cyclooclyl, cyclononyl, bicyclo-[3.1.0]-hexyl, norbornyl, spiro[4.5]decyl, spiro[4.4]nonyl, spiro[4.3]octyl, and spiro[4.2]heptyl. Examples of cycloalkyl with oxo moieties are oxocyclopentyl, and oxocyclobutyl. Cycloalkyl is preferably C₃-C₁₀ cycloalkyl, i.e. cycloalkyl groups containing from 3 to 10 carbon atoms, and more preferably C₃-C₇ cycloalkyl.

The term “aryl”, as used herein, unless otherwise indicated, includes six- to ten-membered carbocyclic aromatic radicals derived from an aromatic hydrocarbon by removal of a hydrogen atom. Aryl furthermore includes bicyclic ring systems. Examples of aryl include, but are not limited to phenyl, naphthyl, indenyl, and fluorenyl.

The terms “heterocyclic” and “heterocyclyl”, as used herein, refer to non-aromatic cyclic groups containing one or more heteroatoms selected from O, S and N. Preferably from one to four heteroatoms, more preferably from one to three heteroatoms. Furthermore, heterocyclic and heterocyclyl includes two-ringed cyclic groups, such as spiro ring systems, fused ring systems and bridged ring systems, wherein said rings share one or two atoms, and wherein at least one of the rings contains a heteroatom selected from O, S, and N. Heterocyclic and heterocyclyl groups also include groups that are substituted with one or more oxo moieties. Examples of heterocyclyl include, but are not limited to aziridinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, quinolizinyl, quinuclidinyl, 1,4-dioxaspiro[4.5]decyl, 1,4-dioxaspiro[4.4]nonyl, 1,4-dioxaspiro[4.3]octyl, and 1,4-dioxaspiro[4.2]heptyl.

The term “Heteroaryl”, as used herein, unless otherwise indicated, refers to aromatic groups containing one or more heteroatoms selected from O, S, and N, preferably from one to four heteroatoms, and more preferably from one to three heteroatoms. Heteroaryl furthermore includes multicyclic groups, wherein at least one ring of the group is aromatic, and at least one of the rings contains a heteroatom selected from O, S, and N. Heteroaryl also include ring systems substituted with one or more oxo moieties. Examples of heteroaryl groups include, but are not limited to pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

The term “linker”, as used herein, refers to a moiety which covalently links different parts of compounds of formula (I), such as the linker L₁ and L₂. Accordingly, one point of the linker may be attached to one part of a compound of (I) and another point of the linker may be attached to another part of said compound. A linker is typically an atom, e.g. —O—, —C—, or —S—, or small moiety such as e.g. —NH— or —NR³—.

The term “Halogen”, as used herein, includes fluoro, chloro, bromo and iodo.

The terms “4-membered”, “5-membered” and “6 membered”, as used herein, refers to ring systems having 4, 5, or 6 non-hydrogen ring atoms, respectively. Examples of 4 membered rings include, but are not limited to, cyclobutane, azetidine, oxetane, oxetane, and thietane. Examples of 5 membered rings include, but are not limited to, cyclopentane, pyrrolidine, tetrahydrofuran, tetrahydrothiophene, and cyclopenta-1,3-diene. Examples of 6 membered rings include, but are not limited to, cyclohexane, piperidine, tetrahydro-2H-pyran, tetrahydro-2H-thiopyran, piperazine, morpholine, cyclohexene, benzene and pyridine.

The term “annulated ring system”, as used herein, refers to ring A and ring B together forms an annulated ring system. The ring system may be any aryl or heteroaryl annulated ring system, including but not limited to, naphthalene, quinoline, isoquinoline, indole, benzotriazole, benzopyrozole, benzimidazole, benzofuran, benzothiophene, benzisoxazole, and benzisothiazole; more specifically the A and B annulated ring system may be selected from the following ring system having the Y moiety attached as indicated 1-naphthalenyl, 4-quinolinyl, 8-quinolinyl, 5-quinolinyl, 8-isoquinolinyl, 7-isoquinolinyl, 4-isoquinolinyl, 3-isoquinolinyl, 1-indolyl, 3-benzofuranyl, 4-benzofuranyl, 7-benzofuranyl, 3-benzothiophenyl, 4-benzothiophenyl, and 7-benzothiophenyl; more preferably the A and B annulated ring system may be selected from 1-naphthalenyl, 8-isoquinolinyl, 1-indolyl, 3-benzofuranyl, and 3-benzothiophenyl; even more preferably the A and B annulated ring system may be selected from 1-naphthalenyl, 8-isoquinolinyl, and 1-indolyl; yet even more preferably the A and B annulated ring system may be selected from the group consisting of 1-naphthalenyl and 1-indolyl.

For compounds of formula (I), A is defined as a 5- or 6-membered aryl or heteroaryl ring. In a preferred embodiment of the invention ring A is an aryl ring. In an alternative preferred embodiment of the invention ring A is a heteroaryl ring; more preferably ring A is a 6-membered heteroaryl ring, such as e.g., a pyridinyl ring annulated to ring B. Alternatively, ring A is a 5-membered heteroaryl ring.

For compounds of formula (I) B is defined as a 4-, 5-, or 6-membered cycloalkyl, aryl, heterocyclyl or heteroaryl ring, which ring together with A forms an annulated ring system. In a preferred embodiment of the invention ring B is a 5-, or 6-membered cycloalkyl, aryl, heterocyclyl or heteroaryl ring; more preferably ring B is a 6-membered cycloalkyl, aryl, or heteroaryl ring; even more preferably ring B is a 6-membered aryl ring, which ring together with A forms an annulated ring system. In alternative preferred embodiment of the invention ring B is a heteroaryl ring; more preferably ring B is a 6-membered heteroaryl ring, which ring together with A forms an annulated ring system. Alternatively, ring B is a 5-membered heteroaryl ring, which ring together with A forms an annulated ring system.

In a preferred embodiment of the invention Y is attached to B in the first position counted from ring B's attachment point to ring A. For example, when rings A and B in combination forms a naphthyl ring, Y is preferably attached in position 1; when A and B in combination forms an indole ring (where B contains the N-atom) then Y preferably is attached in position 1; and when rings A and B in combination forms a quinoline ring, then Y is preferably either attached in the 4 position, 5 position, or 8 position.

Ring A of formula (I) is further defined by X₁ and X₂, which denotes ring atoms of ring A, and may be an atom selected from the group consisting of Carbon, Nitrogen, Oxygen, and Sulphur, wherein at least one of X₁ and X₂ is Carbon. In a preferred embodiment of the invention X₁ and X₂ are each independently an atom selected from the group consisting of Carbon and Nitrogen, wherein at least one of X₁ and X₂ is Carbon; more preferably both of X₁ and X₂ are carbon atoms. In an alternative embodiment one of X₁ and X₂ is a Nitrogen atom, and the other is a Carbon atom.

The size of ring A is furthermore defined by n, which is either 1 or 0, i.e. a 6-membered or a 5 membered ring, respectively. In a preferred embodiment of compounds of formula (I) n is 1.

Examples of appropriate annulated ring systems, i.e. combination of rings A and B, with indicated attachment point for Y are:

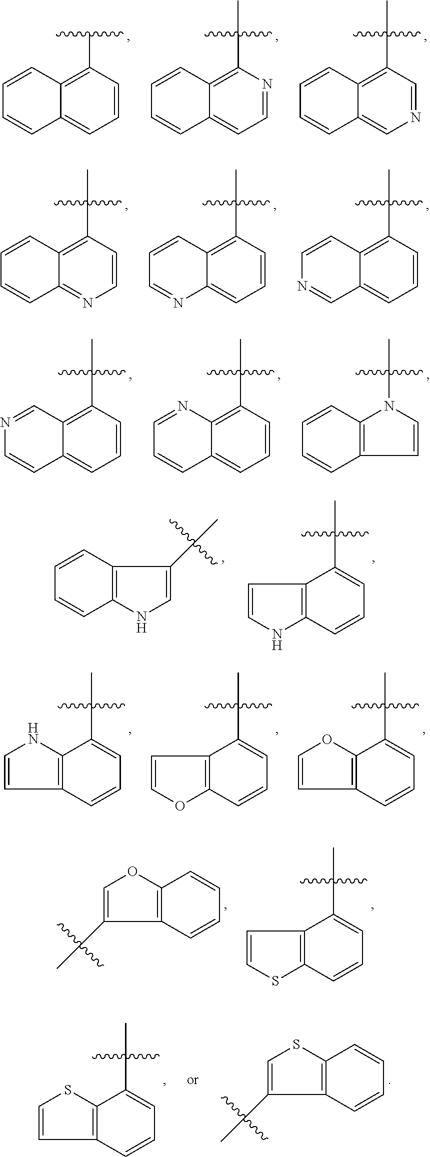

The term “optionally substituted”, as used herein, refers to the optional possibility that one or more hydrogen atoms of a moiety, such as e.g., alkyl, alkenyl, alkynyl, cycloalkyl, aryl, heterocyclyl, heterocyclic ring, and heteroaryl moieties, each independently may or may not be substituted for one or more substituents, preferably 1 to 4 substituents, more preferably 1 to 3 substituents, even more preferably 1 or 2 substituents, and most preferably optionally 1 substituent. For the compounds of formula (I), unless otherwise stated, substituents are selected from the group consisting of halogen, —OH, —SH, —NO₂, —CN, —NH₂, —N₃, C₁₋₆ alkyl, C₁₋₆ alkoxy, C₂₋₆ alkenyl, C₂₋₆ alkynyl, —COOH, —C(O)O—(C₁₋₆ alkyl), —C(O)—NH₂, —C(O)—NH(C₁₋₄ alkyl), —NH(C₁₋₆ alkyl), —N(C₁₋₄ alkyl)(C₁₋₄ alkyl), —NHC(O)—(C₁₋₆ alkyl), —S—(C₁₋₄ alkyl), —S(O)—(C₁₋₄ alkyl), —SO₂—(C₁₋₄ alkyl), —CCl₃, —CF₃, and —CH₂CF₃. In one embodiment of the invention any alkyl, alkenyl, alkynyl, cycloalkyl, aryl, heterocyclyl, and heteroaryl of R¹ and R² each independently optionally is substituted with one or more substituents selected from the group consisting of halogen, —OH, —SH, —NO₂, —CN, —NH₂, —N₃, C₁₋₆ alkyl, C₁₋₆ alkoxy, —COOH, —C(O)O—(C₁₋₆ alkyl), —C(O)—NH₂, —C(O)—NH(C₁₋₄ alkyl), —NH(C₁₋₆ alkyl), —N(C₁₋₄ alkyl)(C₁₋₄ alkyl), —NHC(O)—(C₁₋₆ alkyl), —S—(C₁₋₄ alkyl), —S(O)—(C₁₋₄ alkyl), —SO₂—(C₁₋₄ alkyl), —CCl₃, —CF₃, and —CH₂CF₃. Preferably any alkyl, alkenyl, alkynyl, cycloalkyl, aryl, heterocyclyl, and heteroaryl of R¹ and R² each independently optionally is substituted with one or two substituents, and more preferably optionally substituted with one substituent, of the herein above described substituents.

In a more preferred embodiment of the invention any alkyl, alkenyl, alkynyl, cycloalkyl, aryl, heterocyclyl, and heteroaryl of R¹ and R² each independently optionally is substituted with one or two substituents selected from the group consisting of halogen, —OH, —SH, —NO₂, —CN, —NH₂, —N₃, C₁₋₄ alkyl, C₁₋₄ alkoxy, —COOH, —C(O)O—(C₁₋₄ alkyl), —C(O)—NH₂, —C(O)—NH(C₁₋₄ alkyl), —NH(C₁₋₄ alkyl), —N(C₁₋₄ alkyl)(C₁₋₄ alkyl), —NHC(O)—(C₁₋₄ alkyl), —S—(C₁₋₄ alkyl), —S(O)—(C₁₋₄ alkyl), —SO₂—(C₁₋₄ alkyl), —CCl₃, —CF₃, and —CH₂CF₃. In a specific embodiment of the invention any alkyl, alkenyl, alkynyl, cycloalkyl, aryl, heterocyclyl, and heteroaryl of R¹ and R² each independently optionally is substituted with one or two substituents selected from the group consisting of halogen, —OH, —SH, —NO₂, —CN, —N₃, C₁₋₄ alkyl, C₁₋₄ alkoxy, —COOH, —C(O)O—(C₁₋₄ alkyl), —C(O)—NH₂, —C(O)—NH(C₁₋₄ alkyl), —NHC(O)—(C₁₋₄ alkyl), —S—(C₁₋₄ alkyl), —S(O)—(C₁₋₄ alkyl), —SO₂—(C₁₋₄ alkyl), —CCl₃, —CF₃, and —CH₂CF₃.

In an even more preferred embodiment of the invention any alkyl, alkenyl, alkynyl, cycloalkyl, aryl, heterocyclyl, and heteroaryl of R¹ and R² each independently optionally is substituted with one or two substituents selected from the group consisting of —OH, —CN, —N₃, —CCl₃, —CF₃, and —C(O)O—(C₁₋₂ alkyl). In a yet even more preferred embodiment of the invention any alkyl, alkenyl, alkynyl, cycloalkyl, aryl, heterocyclyl, and heteroaryl of R¹ and R² each independently optionally is substituted with one or two substituents selected from the group consisting of —CN, —N₃, and —CCl₃.

Ring A and ring B of formula (I) may each independently optionally be substituted with one or more substituents selected from the group consisting of halogen, —OH, —SH, —NO₂, —CN, —NH₂, —N₃, C₁₋₄ alkyl, C₁₋₄ alkoxy, C₂₋₄ alkenyl, C₂₋₄ alkynyl, —COOH, —C(O)—NH₂, —NH(C₁₋₄ alkyl), —S—(C₁₋₄ alkyl), —CF₃, and —CH₂CF₃. This applies for any ring A or ring B as mentioned herein for formula (I), and also for compounds of formula (I) where the combination of ring A and ring B has been specified as a specific ring moiety, such as e.g. as a naphthyl moiety, an indolyl moiety, a quinolinyl, or a isoquinolinyl moiety, unless specifically otherwise stated. More preferably ring A and ring B each independently optionally is substituted with one or two substituents, as described herein above; and even more preferably optionally with one substituent.

In a preferred embodiment of compounds of formula (I) ring A and ring B each independently optionally is substituted with one or more substituents selected from the group consisting of halogen, —OH, —CN, —N₃, C₁₋₄ alkyl, C₁₋₄ alkoxy, and —CF₃. In a more specific embodiment ring A and ring B each independently optionally is substituted with one or more substituents selected from the group consisting of bromo, iodo, fluoro, chloro, —OH, —CN, —N₃, methyl, ethyl, propyl, isopropyl, butyl, iso-butyl, sec-butyl, tert-butyl, methoxy, ethoxy, propoxy, isopropoxy, butoxy, isobutoxy, sec-butoxy, tert-butoxy and —CF₃. In an even more specific embodiment ring A and ring B each independently optionally is substituted with one or more substituents selected from the group consisting of bromo, iodo, fluoro, chloro, —OH, —CN, —N₃, methyl, methoxy, and —CF₃; yet even more specifically the group consisting of bromo, iodo, fluoro, chloro, —CN, —N₃, methyl, and methoxy; yet even more specifically fluoro, chloro, methyl, and methoxy.

In one embodiment of compounds of formula (I) Y is

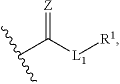

wherein Z is an atom selected from the group consisting of oxygen and sulphur, and L₁ and R¹ is as defined herein above. In a preferred embodiment Z is an Oxygen atom, and Y is then

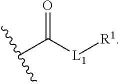

Alternatively, Z is a Sulphur atom and Y is then

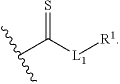

For compounds of formula (I), unless otherwise stated, L₁ is a linker selected from the group consisting of —O—, —NH—, —NR³—, and —C—; more preferably L₁ is selected from the group consisting of —O—, —NH—, and —NR³—. In a specific embodiment of the compounds according to the present invention L₁ is —O—. In an alternative embodiment L₁ is selected from the group consisting of —NH— and —NR³—.

For compounds of formula (I), unless otherwise stated, R¹ is selected from the group consisting of C₁₋₈ alkyl, C₁₋₈ alkoxy, C₂₋₈ alkenyl, C₂₋₈ alkynyl, C₃₋₁₀ cycloalkyl, aryl, heterocyclyl, heteroaryl, —NH—C₁₋₆ alkyl, C₃₋₁₀ cycloalkyl-C₁₋₆ alkyl, aryl-C₁₋₆ alkyl, heterocyclyl-C₁₋₆ alkyl, and heteroaryl-C₁₋₆ alkyl; more preferably R¹ is selected from the group consisting of C₁₋₆ alkyl, C₁₋₆ alkoxy, C₂₋₆ alkenyl, C₂₋₆ alkynyl, C₃₋₅ cycloalkyl, aryl, heterocyclyl, heteroaryl, —NH—C₁₋₆ alkyl, C₃₋₅ cycloalkyl-C₁₋₆ alkyl, aryl-C₁₋₆ alkyl, heterocyclyl-C₁₋₆ alkyl, and heteroaryl-C₁₋₆ alkyl, where any of these optionally is substituted with one or more substituents, as defined herein above.

In a specific embodiment of compounds of formula (I) R¹ is selected from the group consisting of C₁₋₆ alkyl, C₁₋₆ alkoxy, C₂₋₆ alkenyl, C₂₋₆ alkynyl, C₃₋₅ cycloalkyl, aryl, heterocyclyl, heteroaryl, —NH—C₁₋₆ alkyl, C₃₋₅ cycloalkyl-C₁₋₆ alkyl, aryl-C₁₋₆ alkyl, and heterocyclyl-C₁₋₆ alkyl, where any of these optionally is substituted with one or more substituents, as defined herein above. In a more specific embodiment of compounds of formula (I) R¹ is selected from the group consisting of C₁₋₆ alkyl, C₁₋₆ alkoxy, C₂₋₆ alkenyl, C₂₋₆ alkynyl, C₃₋₅ cycloalkyl, aryl, heterocyclyl, heteroaryl, and —NH—C₁₋₆ alkyl, where any of these optionally is substituted with one or more substituents, as defined herein above.

In a preferred embodiment of the compounds of formula (I) R¹ is selected from the group consisting of C₁₋₆ alkyl, C₁₋₆ alkoxy, C₂₋₆ alkenyl, C₂₋₆ alkynyl, C₃₋₅ cycloalkyl, and —NH—C₁₋₆ alkyl, where any of these optionally is substituted with one or more substituents. In a more preferred embodiment of the compounds of formula (I) R¹ is selected from the group consisting of C₁₋₆ alkyl, C₁₋₆ alkoxy, C₂₋₆ alkenyl, C₂₋₆ alkynyl, and —NH—C₁₋₆ alkyl, where any of these optionally is substituted with one or more substituents. In an even more preferred embodiment of the compounds of formula (I) R¹ is selected from the group consisting of C₁₋₄ alkyl, C₁₋₄ alkoxy, C₂₋₄ alkenyl, C₂₋₄ alkynyl, and —NH—C₁₋₄ alkyl, where any of these optionally is substituted with one or more substituents. In a yet even more preferred embodiment R¹ is selected from the group consisting of C₁₋₄ alkyl and C₂₋₄ alkenyl, wherein any of these optionally is substituted with one or more substituents, as defined herein above. In a yet even more preferred embodiment R¹ is C₁₋₄ alkyl, wherein the alkyl optionally is substituted with one or more substituents, as defined herein above.

For compounds of formula (I), unless otherwise stated, R³ is selected from the group consisting of C₁₋₆ alkyl, C₂₋₆ alkenyl, and C₂₋₆ alkynyl; more preferably R³ is selected from the group consisting of C₁₋₄ alkyl, C₂₋₄ alkenyl, and C₂₋₄ alkynyl; even more preferably R³ is C₁₋₄ alkyl. In a specific embodiment of the compounds of formula (I) R³ is selected from the group consisting of methyl, ethyl, propyl, isopropyl, butyl, iso-butyl, sec-butyl, and tert-butyl; more preferably R³ is selected from the group consisting of methyl and ethyl; even more preferably R³ is methyl.

In a specific embodiment of the compounds of formula (I) L₁ is —NH— or —NR³— and R¹ is selected from the group consisting of C₁₋₆ alkyl, C₁₋₆ alkoxy, C₂₋₆ alkenyl, C₂₋₆ alkynyl, C₃₋₅ cycloalkyl, aryl, heterocyclyl, heteroaryl, —NH—C₁₋₆ alkyl, C₃₋₅ cycloalkyl-C₁₋₆ alkyl, aryl-C₁₋₆ alkyl, and heterocyclyl-C₁₋₆ alkyl, where any of these optionally is substituted with one or more substituents, as defined herein above. In a more specific embodiment of the compounds of formula (I) L₁ is —NH—, —NR³— and R¹ is selected from the group consisting of C₁₋₆ alkyl, C₁₋₆ alkoxy, C₂₋₆ alkenyl, C₂₋₆ alkynyl, C₃₋₅ cycloalkyl, aryl, heterocyclyl, heteroaryl, and —NH—C₁₋₆ alkyl, where any of these optionally is substituted with one or more substituents, as defined herein above.

In another embodiment of compounds of formula (I) Y is

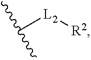

wherein L₂ and R² is as defined herein above.

For compounds of formula (I), unless otherwise stated, L₂ is a linker selected from the group consisting of —O—, —S—, —NH—, and —NR⁴—; preferably L₂ is selected from the group consisting of —O— and —S—. In a specific embodiment of the compounds according to the present invention L₂ is —O—. In an alternative embodiment L₂ is selected from the group consisting of —S—, —NH— and —NR⁴; more preferably L₂ is —S—. Alternatively, L₂ is selected from the group consisting of —NH— and —NR⁴.

For compounds of formula (I), unless otherwise stated, R² is selected from the group consisting of C₁₋₈ alkyl, C₁₋₈ alkoxy, C₂₋₈ alkenyl, C₂₋₈ alkynyl, C₃₋₁₀ cycloalkyl, aryl, heterocyclyl, heteroaryl, —NH—C₁₋₆ alkyl, C₃₋₁₀ cycloalkyl-C₁₋₆ alkyl, aryl-C₁₋₆ alkyl, heterocyclyl-C₁₋₆ alkyl, and heteroaryl-C₁₋₆ alkyl; more preferably R² is selected from the group consisting of C₁₋₆ alkyl, C₂₋₆ alkenyl, C₂₋₆ alkynyl, C₃₋₅ cycloalkyl, aryl, heterocyclyl, heteroaryl, C₃₋₅ cycloalkyl-C₁₋₆ alkyl, aryl-C₁₋₆ alkyl, heterocyclyl-C₁₋₆ alkyl, and heteroaryl-C₁₋₆ alkyl, where any of these optionally is substituted with one or more substituents, as defined herein above.

In a specific embodiment of compounds of formula (I) R² is selected from the group consisting of C₁₋₆ alkyl, C₂₋₆ alkenyl, C₂₋₆ alkynyl, C₃₋₅ cycloalkyl, aryl, heterocyclyl, heteroaryl, C₃₋₅ cycloalkyl-C₁₋₆ alkyl, aryl-C₁₋₆alkyl, heterocyclyl-C₁₋₆ alkyl, and heteroaryl-C₁₋₆ alkyl, where any of these optionally is substituted with one or more substituents, with the proviso that C₁₋₆ alkyl is not substituted with an methylamino group when L₂ is —O—. In another specific embodiment of compounds of formula (I) R² is selected from the group consisting of C₁₋₆ alkyl, C₂₋₆ alkenyl, C₂₋₆ alkynyl, C₃₋₅ cycloalkyl, aryl, heterocyclyl, heteroaryl, C₃₋₅ cycloalkyl-C₁₋₆ alkyl, aryl-C₁₋₆ alkyl, and heterocyclyl-C₁₋₆ alkyl, where any of these optionally is substituted with one or more substituents, as defined herein above; even more specifically R² is selected from the group consisting of C₁₋₆ alkyl, C₂₋₆ alkenyl, C₂₋₆ alkynyl, C₃₋₅ cycloalkyl, aryl, heterocyclyl, and heteroaryl, where any of these optionally is substituted with one or more substituents, as defined herein above.

In a preferred embodiment of the compounds of formula (I) R² is selected from the group consisting of C₁₋₆ alkyl, C₂₋₆ alkenyl, C₂₋₆ alkynyl, and C₃₋₅ cycloalkyl; more preferably R² is selected from the group consisting of C₁₋₄ alkyl, C₂₋₄ alkenyl, C₂₋₄ alkynyl, and C₃₋₅ cycloalkyl; even more preferably R² is selected from the group consisting of C₁₋₄ alkyl, C₂₋₄ alkenyl, and C₂₋₄ alkynyl, where any of these optionally is substituted with one or more substituents, as defined herein above. In a yet even more preferred embodiment R² is selected from the group consisting of C₁₋₄ alkyl and C₂₋₄ alkenyl, where any of these optionally is substituted with one or more substituents, as defined herein above. In a yet even more preferred embodiment R² is C₁₋₄ alkyl, wherein the alkyl optionally is substituted with one or more substituents.

For compounds of formula (I), unless otherwise stated, R⁴ is selected from the group consisting of C₁₋₆ alkyl, C₂₋₆ alkenyl, and C₂₋₆ alkynyl; more preferably R⁴ is selected from the group consisting of C₁₋₄ alkyl, C₂₋₄ alkenyl, and C₂₋₄ alkynyl; even more preferably R⁴ is C₁₋₄ alkyl. In a specific embodiment of the compounds of formula (I) R⁴ is selected from the group consisting of methyl, ethyl, propyl, isopropyl, butyl, iso-butyl, sec-butyl, and tert-butyl; more preferably R⁴ is selected from the group consisting of methyl and ethyl; even more preferably R³ is methyl.

In a specific embodiment of the compounds of formula (I) L₂ is —O— and R² is selected from the group consisting of C₁₋₆ alkyl, C₂₋₆ alkenyl, C₂₋₆ alkynyl, C₃₋₅ cycloalkyl, aryl, heterocyclyl, heteroaryl, C₃₋₅ cycloalkyl-C₁₋₆ alkyl, aryl-C₁₋₆ alkyl, heterocyclyl-C₁₋₆ alkyl, and heteroaryl-C₁₋₆ alkyl, where any of these optionally is substituted with one or more substituents, with the proviso that C₁₋₆ alkyl is not substituted with an methylamino group.

In another specific embodiment of the compounds of formula (I) L₂ is —O— and R² is selected from the group consisting of C₁₋₆ alkyl, C₂₋₆ alkenyl, C₂₋₆ alkynyl, C₃₋₅ cycloalkyl, aryl, heterocyclyl, heteroaryl, C₃₋₅ cycloalkyl-C₁₋₆ alkyl, aryl-C₁₋₆ alkyl, and heterocyclyl-C₁₋₆ alkyl, where any of these optionally is substituted with one or more substituents, as defined herein above.

Examples of preferred compounds of formula (I) are:

1-Naphthoic acid methyl ester;

1-Naphthoic acid ethyl ester;

1-Naphthoic acid isopropyl ester;

1-Naphthoic acid propyl ester;

1-Naphthoic acid 2-hydroxyethyl ester;

1-Naphthoic acid ethylamide;

1-Naphthoic acid pentyl ester;

1-Naphthoic acid 2-propenyl ester;

1-Naphthoic acid 2-propynyl ester;

1-Naphthoic acid secbutyl ester;

1-Naphthoic acid cyclopropylmethyl ester;

1-Naphthoic acid cyclopentyl ester;

1-Naphthoic acid cyclohexyl ester;

1-Naphthoic acid-2-methoxyethyl ester;

1-Naphthoic acid-2-methylsulfanyl ester;

(±) 1,2,3,4-Tetrahydro-1-naphthoic acid ethyl ester;

Benzotriazole-1-carboxylic acid ethyl ester;

2,3-Dihydro-indole-1-carboxylic acid ethyl ester;

Indole-1-carboxylic acid ethyl ester;

4-Methyl-indole-1-carboxylic acid ethyl ester;

5-Methyl-indole-1-carboxylic acid ethyl ester;

6-Methyl-indole-1-carboxylic acid ethyl ester;

6-Chloro-indole-1-carboxylic acid ethyl ester;

3-Methyl-indole-1-carboxylic acid ethyl ester;

7-Methyl-indole-1-carboxylic acid ethyl ester;

4-Chloro-indole-1-carboxylic acid ethyl ester;

7-Chloro-indole-1-carboxylic acid ethyl ester;

Indole-1-carboxylic acid 1,1,1-trichloroethyl ester;

7-Hydroxy-indole-1-carboxylic acid ethyl ester;

6-Hydroxy-indole-1-carboxylic acid ethyl ester;

5-Hydroxy-indole-1-carboxylic acid ethyl ester;

4-Hydroxy-indole-1-carboxylic acid ethyl ester;

7-Methoxy-indole-1-carboxylic acid ethyl ester;

6-Methoxy-indole-1-carboxylic acid ethyl ester;

5-Methoxy-indole-1-carboxylic acid ethyl ester;

4-Methoxy-indole-1-carboxylic acid ethyl ester;

5-Chloro-indole-1-carboxylic acid ethyl ester;

Indole-1-carboxylic acid ethyl amide;

Indole-1-carbothioic acid ethyl amide;

1-Propoxy-naphthalene;

Methyl-[1]naphthyl sulphide;

Ethyl-[1]naphthyl sulphide;

1-Propyl-sulfanyl-1-naphthalene;

1-Butyl-sulfanyl-1-naphthalene;

3-Ethenyl-sulfanyl-1-naphthalene;

3-Ethynyl-sulfanyl-1-naphthalene;

2-Hydroxyethyl-1-naphthyl sulphide;

3-Hydroxypropyl-1-naphthyl sulphide;

3-Dimethylaminopropyl-1-naphthyl sulphide;

Methyl β-(1-naphthylthio)-propionate;

β-(1-Naphthylthio)-propionic acid;

(Naphthalen-1-ylsulfanyl)-acetonitrile;

2-Dimethylaminoethyl-1-naphthyl sulphide;

Isopropyl-1-naphthyl sulphide;

sec-Butyl-1-naphthyl sulfide;

isobutyl-1-naphthyl sulfide;

Cyclohexyl-1-naphthyl sulphide;

Cyclopentyl-1-naphthyl sulphide;

(2-Methoxy-ethylsulfanyl)-1-naphthalene;

7-Ethylsulfanyl-1H-indole;

7-Ethylsulfanyl-benzofuran;

4-Ethylsulfanyl-1H-indole; and

4-Ethylsulfanyl-benzofuran.

In a preferred embodiment of the invention specific compounds of formula (I) are:

1-Naphthoic acid methyl ester;

1-Naphthoic acid ethyl ester;

1-Naphthoic acid isopropyl ester;

1-Naphthoic acid propyl ester;

1-Naphthoic acid 2-hydroxyethyl ester;

1-Naphthoic acid ethylamide;

1-Naphthoic acid pentyl ester;

1-Naphthoic acid 2-propenyl ester;

1-Naphthoic acid 2-propynyl ester;

1-Naphthoic acid secbutyl ester;

1-Naphthoic acid cyclopropylmethyl ester;

1-Naphthoic acid cyclopentyl ester;

1-Naphthoic acid cyclohexyl ester;

(±) 1,2,3,4-Tetrahydro-1-naphthoic acid ethyl ester;

Benzotriazole-1-carboxylic acid ethyl ester;

2,3-Dihydro-indole-1-carboxylic acid ethyl ester;

Indole-1-carboxylic acid ethyl ester;

4-Methyl-indole-1-carboxylic acid ethyl ester;

5-Methyl-indole-1-carboxylic acid ethyl ester;

6-Methyl-indole-1-carboxylic acid ethyl ester;

6-Chloro-indole-1-carboxylic acid ethyl ester;

3-Methyl-indole-1-carboxylic acid ethyl ester;

7-Methyl-indole-1-carboxylic acid ethyl ester;

4-Chloro-indole-1-carboxylic acid ethyl ester;

7-Chloro-indole-1-carboxylic acid ethyl ester;

Indole-1-carboxylic acid 1,1,1-trichloroethyl ester;

7-Hydroxy-indole-1-carboxylic acid ethyl ester;

6-Hydroxy-indole-1-carboxylic acid ethyl ester;

5-Hydroxy-indole-1-carboxylic acid ethyl ester;

4-Hydroxy-indole-1-carboxylic acid ethyl ester;

7-Methoxy-indole-1-carboxylic acid ethyl ester;

6-Methoxy-indole-1-carboxylic acid ethyl ester;

5-Methoxy-indole-1-carboxylic acid ethyl ester;

4-Methoxy-indole-1-carboxylic acid ethyl ester;

5-Chloro-indole-1-carboxylic acid ethyl ester;

1-Propoxy-naphthalene;

Methyl-[1]naphthyl sulphide;

Ethyl-[1]naphthyl sulphide;

1-Propyl-sulfanyl-1-naphthalene;

1-Butyl-sulfanyl-1-naphthalene;

3-Ethenyl-sulfanyl-1-naphthalene;

3-Ethynyl-sulfanyl-1-naphthalene;

2-Hydroxyethyl-1-naphthyl sulphide;

3-Hydroxypropyl-1-naphthyl sulphide;

3-Dimethylaminopropyl-1-naphthyl sulphide;

Methyl β-(1-naphthylthio)-propionate;

β-(1-Naphthylthio)-propionic acid;

(Naphthalen-1-ylsulfanyl)-acetonitrile;

2-Dimethylaminoethyl-1-naphthyl sulphide;

Isopropyl-1-naphthyl sulphide;

sec-Butyl-1-naphthyl sulfide;

isobutyl-1-naphthyl sulfide;

Cyclohexyl-1-naphthyl sulphide; and

Cyclopentyl-1-naphthyl sulphide.

In a more preferred embodiment of the invention specific compounds of formula (I) are:

1-Naphthoic acid methyl ester;

1-Naphthoic acid ethyl ester;

1-Naphthoic acid isopropyl ester;

1-Naphthoic acid propyl ester;

1-Naphthoic acid 2-hydroxyethyl ester;

1-Naphthoic acid ethylamide;

1-Naphthoic acid pentyl ester;

1-Naphthoic acid 2-propenyl ester;

1-Naphthoic acid 2-propynyl ester;

1-Naphthoic acid secbutyl ester;

1-Naphthoic acid cyclopropylmethyl ester;

1-Naphthoic acid cyclopentyl ester;

1-Naphthoic acid cyclohexyl ester;

(±) 1,2,3,4-Tetrahydro-1-naphthoic acid ethyl ester;

Benzotriazole-1-carboxylic acid ethyl ester;

2,3-Dihydro-indole-1-carboxylic acid ethyl ester;

Indole-1-carboxylic acid ethyl ester;

4-Methyl-indole-1-carboxylic acid ethyl ester;

5-Methyl-indole-1-carboxylic acid ethyl ester;

6-Methyl-indole-1-carboxylic acid ethyl ester;

6-Chloro-indole-1-carboxylic acid ethyl ester;

3-Methyl-indole-1-carboxylic acid ethyl ester;

7-Methyl-indole-1-carboxylic acid ethyl ester;

4-Chloro-indole-1-carboxylic acid ethyl ester;

7-Chloro-indole-1-carboxylic acid ethyl ester;

1-Propoxy-naphthalene;

Methyl-[1]naphthyl sulphide;

Ethyl-[1]naphthyl sulphide;

1-Propyl-sulfanyl-1-naphthalene;

1-Butyl-sulfanyl-1-naphthalene;

3-Ethenyl-sulfanyl-1-naphthalene;

3-Ethynyl-sulfanyl-1-naphthalene;

2-Hydroxyethyl-1-naphthyl sulphide;

3-Hydroxypropyl-1-naphthyl sulphide;

3-Dimethylaminopropyl-1-naphthyl sulphide;

Methyl β-(1-naphthylthio)-propionate;

β-(1-Naphthylthio)-propionic acid; and

(Naphthalen-1-ylsulfanyl)-acetonitrile.

In an even more preferred embodiment of the invention specific compounds of formula (I) are:

1-Naphthoic acid methyl ester;

1-Naphthoic acid ethyl ester;

1-Naphthoic acid isopropyl ester;

1-Naphthoic acid propyl ester;

1-Naphthoic acid 2-hydroxyethyl ester;

Indole-1-carboxylic acid ethyl ester;

Ethyl-[1]naphthyl sulfide;

1-Propyl-sulfanyl-1-naphthalene;

3-Ethenyl-sulfanyl-1-naphthalene;

3-Ethynyl-sulfanyl-1-naphthalene; and

(Naphthalen-1-ylsulfanyl)-acetonitrile.

The terms “treating” and “treatment”, as used herein, refers to reversing, alleviating, inhibiting the progress of, or preventing the disorder or condition to which such term applies, or one or more symptoms of such disorder or condition.

The term “protecting group”, as used herein, means a hydroxy or amino protecting group which is selected from typical hydroxy or amino protecting groups described in Protective Groups in Organic Synthesis edited by T. W. Greene et al. (John Wiley & Sons, 1991).

The term “psychiatric medication”, as used herein, refers to active substances having an effect on CNS disorders, including but not limited to, known and future licensed psychoactive drugs.

The term “pharmaceutical acceptable salt, solvate or prodrug” as used herein refers to those acid and base additions salts, solvates, and prodrugs of the compounds of the present invention which are, within the scope of sound medical judgment, suitable for use without undue toxicity, irritation, allergic response, and the like, commensurate with a reasonable benefit/risk ratio, and effective for their intended use, as well as the zwitterionic forms, where possible, of the compounds of the invention.

Pharmaceutically acceptable acid and base addition salts refers to the relatively non-toxic, inorganic and organic addition salts of compounds of the present invention. These salts can be prepared in situ during the final isolation and purification of the compounds or by separately reacting the purified compound in its free acid or base form with a suitable organic or inorganic compound and isolating the salt thus formed. In so far as the compounds of formula (I) of this invention are basic compounds, they are all capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt.

The pharmaceutically acceptable acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metal hydroxides, or of organic amines. Examples of metals used as cations are sodium, potassium, magnesium, calcium, and the like. Examples of suitable amines are N,N′-dibenzylethylenediamine, chloroprocaine, choline, diethanolamine, ethylenediamine, N-methylglucamine, and procaine. The base addition salts of acidic compounds are prepared by contacting the free acid form with a sufficient amount of the desired base to produce the salt in the conventional manner. The free acid form may be regenerated by contacting the salt form with an acid and isolating the free acid in a conventional manner. The free acid forms differ from their respective salt forms somewhat in certain physical properties such as solubility in polar solvents, but otherwise the salts are equivalent to their respective free acid for purposes of the present invention.

Salts may be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts may also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts may include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. (See, for example, Berge S. M. et al., “Pharmaceutical Salts,” J. Pharm. Sci., 1977; 66:1-19 which is incorporated herein by reference.)

The compounds of the present invention can exist in unsolvated forms as well as solvated forms, including hydrated forms. In general, the solvated forms, including hydrated forms, are equivalent to unsolvated forms and are intended to be encompassed within the scope of the present invention.

The term “prodrug” refers to compounds that are rapidly transformed in vivo to yield the parent compound of the above formulae, for example, by hydrolysis in blood. A thorough discussion is provided in T. Higuchi and V Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference. Examples of prodrugs include pharmaceutically acceptable, non-toxic esters of the compounds of the present invention, including C₁-C₆ alkyl esters wherein the alkyl group is a straight or branched chain. Acceptable esters also include C₅-C₇ cycloalkyl esters as well as arylalkyl esters such as, but not limited to benzyl. C₁-C₄ alkyl esters are preferred. Esters of the compounds of the present invention may be prepared according to conventional methods “March's Advanced Organic Chemistry, 5^{th} Edition”. M. B. Smith & J. March, John Wiley & Sons, 2001.

Compounds of formula (I) may contain chiral centers and therefore may exist in different enantiomeric and diastereomeric forms. This invention relates to all optical isomers and all stereoisomers of compounds of the formula (I), both as racemic mixtures and as individual enantiomers and diastereoisomers ((+)- and (−)-optically active forms) of such compounds, and mixtures thereof, and to all pharmaceutical compositions and methods of treatment defined below that contain or employ them, respectively. Individual isomers can be obtained by known methods, such as optical resolution, optically selective reaction, or chromatographic separation in the preparation of the final product or its intermediate.

The present invention also includes isotopically-labelled compounds, which are identical to those recited in formula (I), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine, iodine, and chlorine, such as ³H, ¹¹C, ¹⁴C, ¹⁸F, ¹²³I and ¹²⁵I. Compounds of the present invention and pharmaceutically acceptable salts of said compounds that contain the aforementioned isotopes and/or other isotopes of other atoms are within the scope of this invention. Isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula (I) of the present invention can generally be prepared by carrying out the procedures disclosed in the synthesis Schemes and/or in the Examples below, by substituting a readily available isotopically labelled reagent for a non-isotopically labelled reagent.

Compounds of Formula (I) Having Formulas (Ia), (Ib), (II), (IIa), (IIb), (IIc), (III), (IIIa), (IV), (V), and (VI)

The present invention relates to compounds of formula (I), the use of these compounds as medicaments, and especially these compounds for treating a CNS disorder, as described herein above. The following compounds defined by formulas (Ia), (Ib), (II), (IIa), (IIb), (IIc), (III), (IIIa), (IV), (V), and (VI) all fall within the definition of compounds of formula (I), therefore every aspect of the present invention which applies for compounds of formula (I) like wise applies for compounds of formulas (Ia), (Ib), (II), (IIa), (IIb), (IIc), (III), (IIIa), (IV), (V), and (VI), mutatis mutandis.

In one embodiment of the invention the compounds of formula (I) are of formula (Ia)

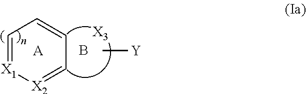

or a pharmaceutically acceptable salt, solvate or prodrug thereof,

wherein X₃ is an atom selected from the group consisting of Carbon, Nitrogen, Oxygen, and Sulphur; and wherein A, B, X₁, X₂, Y, n, Z, L₁, L₂, R¹, R², R³, and R⁴ are as defined for formula (I) herein above.

As used herein X₃ is intended to represent any of the ring atoms of ring B, including the ring atom to which moiety Y is attached. For example if B is an indole ring, X₃ is a nitrogen atom, and Y is preferably attached to the nitrogen atom of the indole ring.

In a preferred embodiment of formula (Ia) X₃ is an atom selected from carbon and nitrogen. In one embodiment X₃ is nitrogen, and in another embodiment X₃ is carbon.

In a more specific embodiment of formula (I) and (Ia) the compounds are of formula (Ib)

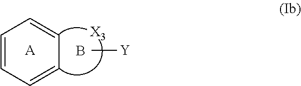

or a pharmaceutically acceptable salt, solvate or prodrug thereof,

wherein A, B, X₃, Y, n, Z, L₁, L₂, R¹, R², R³, and R⁴ are as defined for formula (I) and (Ia) herein above, and where rings A and B independently optionally is substituted with one or more substituents as described above for formula (I).

In a further embodiment of the invention the compounds of formula (I) are of formula (II)

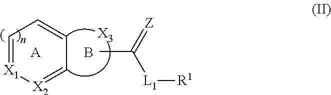

or a pharmaceutically acceptable salt, solvate or prodrug thereof,

wherein A, B, X₁, X₂, X₃, n, Z, L₁, R¹, and R³ are as defined for formula (I) and formula (Ia) herein above.

In a more specific embodiment of formula (I) and (II) the compounds are of formula (IIa)

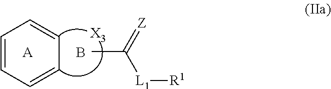

or a pharmaceutically acceptable salt, solvate or prodrug thereof,

wherein A, B, X₃, Z, L₁, R¹, and R³ are as defined for formula (I), (Ia), and (II) herein above, and where rings A and B independently optionally is substituted with one or more substituents as described above for formula (I).

In an even more specific embodiment of formula (I) and (II) the compounds are of formula (IIb)

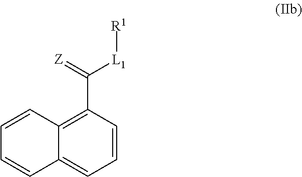

or a pharmaceutically acceptable salt, solvate or prodrug thereof,

wherein Z, L₁, R¹, and R³ are as defined for formula (I) and (II) herein above, and where rings A and B, here specified as a naphthyl moiety, independently optionally is substituted with one or more substituents as described above for formula (I).

In one embodiment of the compounds of formulas (I), (II), (IIa) or (IIb) L₁ and R¹ is as defined for formula (I) herein above, with the proviso that when L₁ is —NH—, then R¹ is not heteroaryl-C₁₋₆ alkyl, and/or with the proviso that when L₁ is —C—, then R¹ is not heteroaryl-C₁₋₆ alkyl.

In another more specific embodiment of formula (I) and (II) the compounds are of formula (IIc)

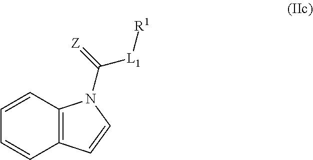

or a pharmaceutically acceptable salt, solvate or prodrug thereof,

wherein Z, L₁, R¹, and R³ are as defined for formula (I) and (II) herein above, and where rings A and B, here specified as an indolyl moiety, independently optionally is substituted with one or more substituents as described above for formula (I).

In a further embodiment of the invention the compounds of formula (I) are of formula (III)

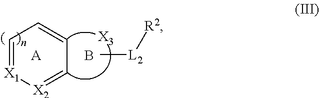

or a pharmaceutically acceptable salt, solvate or prodrug thereof,

wherein A, B, X₁, X₂, X₃, n, Z, L₂, R², and R⁴ are as defined for formula (I) and formula (Ia) herein above.

In a more specific embodiment of formula (I) and (III) the compounds are of formula (IIIa)

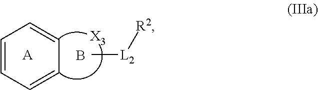

or a pharmaceutically acceptable salt, solvate or prodrug thereof,

wherein A, B, X₃, Z, L₂, R², and R⁴ are as defined for formula (I), (Ia), and (III) herein above, and where rings A and B independently optionally is substituted with one or more substituents as described above for formula (I).

In one embodiment of the compounds of formulas (I), (III), or (IIIa) L₂ and R² are as defined for formula (I) herein above, with the proviso that when L₂ is —O—, then R² is not heteroaryl-C₁₋₆ alkyl, and/or with the proviso that when L₂ is —O—, then R² is not C₁₋₈ alkyl substituted with a methylamino group.

In a preferred embodiment of the compounds of formulas (I), (III), or (IIIa) L₂ is as defined for formula (I) herein above, and R² is selected from the group consisting of C₁₋₆ alkyl, C₂₋₆ alkenyl, C₂₋₆ alkynyl, C₃₋₅ cycloalkyl, aryl, heterocyclyl, and heteroaryl, where any of these optionally is substituted with one or more substituents, with the proviso that when L₂ is —O—, then R² is not C₁₋₆ alkyl substituted with a methylamino group.

In a further embodiment of the invention the compounds of formula (I) are of formula (IV)

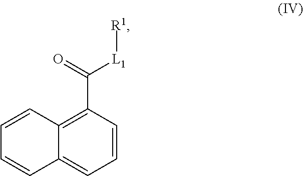

or a pharmaceutically acceptable salt, solvate or prodrug thereof,

wherein L₁, R¹, and R³ are as defined for formula (I) herein above, and where rings A and B, here specified as a naphthyl moiety, independently optionally is substituted with one or more substituents as described above for formula (I).

In a further embodiment of the invention the compounds of formula (I) are of formula (V)

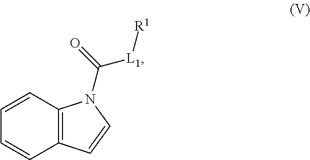

or a pharmaceutically acceptable salt, solvate or prodrug thereof,

wherein L₁, R¹, and R³ are as defined for formula (I) herein above, and where rings A and B, here specified as an indolyl moiety, independently optionally is substituted with one or more substituents as described above for formula (I).

In a further embodiment of the invention the compounds of formula (I) are of formula (VI)

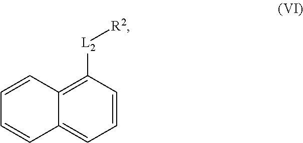

or a pharmaceutically acceptable salt, solvate or prodrug thereof,

wherein L₂, R², and R⁴ are as defined for formula (I) herein above, and where rings A and B, here specified as a naphthyl moiety, independently optionally is substituted with one or more substituents as described above for formula (I).

A preferred embodiment of the present invention relates to compounds of formulas (I), (Ia), (Ib), (II), or (IIa), selected from the group consisting of:

1-Naphthoic acid methyl ester;

1-Naphthoic acid ethyl ester;

1-Naphthoic acid isopropyl ester;

1-Naphthoic acid propyl ester;

1-Naphthoic acid 2-hydroxyethyl ester;

1-Naphthoic acid ethylamide;

1-Naphthoic acid pentyl ester;

1-Naphthoic acid 2-propenyl ester;

1-Naphthoic acid 2-propynyl ester;

1-Naphthoic acid secbutyl ester;

1-Naphthoic acid cyclopropylmethyl ester;

1-Naphthoic acid cyclopentyl ester;

1-Naphthoic acid cyclohexyl ester;

1-Naphthoic acid-2-methoxyethyl ester;

1-Naphthoic acid-2-methylsulfanyl ester;

(±) 1,2,3,4-Tetrahydro-1-naphthoic acid ethyl ester;

Benzotriazole-1-carboxylic acid ethyl ester;

2,3-Dihydro-indole-1-carboxylic acid ethyl ester;

Indole-1-carboxylic acid ethyl ester;

4-Methyl-indole-1-carboxylic acid ethyl ester;

5-Methyl-indole-1-carboxylic acid ethyl ester;

6-Methyl-indole-1-carboxylic acid ethyl ester;

6-Chloro-indole-1-carboxylic acid ethyl ester;

3-Methyl-indole-1-carboxylic acid ethyl ester;

7-Methyl-indole-1-carboxylic acid ethyl ester;

4-Chloro-indole-1-carboxylic acid ethyl ester;

7-Chloro-indole-1-carboxylic acid ethyl ester;

Indole-1-carboxylic acid 1,1,1-trichloroethyl ester;

7-Hydroxy-indole-1-carboxylic acid ethyl ester;

6-Hydroxy-indole-1-carboxylic acid ethyl ester;

5-Hydroxy-indole-1-carboxylic acid ethyl ester;

4-Hydroxy-indole-1-carboxylic acid ethyl ester;

7-Methoxy-indole-1-carboxylic acid ethyl ester;

6-Methoxy-indole-1-carboxylic acid ethyl ester;

5-Methoxy-indole-1-carboxylic acid ethyl ester;

4-Methoxy-indole-1-carboxylic acid ethyl ester;

5-Chloro-indole-1-carboxylic acid ethyl ester;

Indole-1-carboxylic acid ethyl amide; and

Indole-1-carbothioic acid ethyl amide.

A more preferred embodiment of the present invention relates to compounds of formulas (I), (Ia), (Ib), (II), or (IIa), selected from the group consisting of:

1-Naphthoic acid methyl ester;

1-Naphthoic acid ethyl ester;

1-Naphthoic acid isopropyl ester;

1-Naphthoic acid propyl ester;

1-Naphthoic acid 2-hydroxyethyl ester;

1-Naphthoic acid ethylamide;

1-Naphthoic acid pentyl ester;

1-Naphthoic acid 2-propenyl ester;

1-Naphthoic acid 2-propynyl ester;

1-Naphthoic acid secbutyl ester;

1-Naphthoic acid cyclopropylmethyl ester;

1-Naphthoic acid cyclopentyl ester;

1-Naphthoic acid cyclohexyl ester;

(±) 1,2,3,4-Tetrahydro-1-naphthoic acid ethyl ester;

Benzotriazole-1-carboxylic acid ethyl ester;

2,3-Dihydro-indole-1-carboxylic acid ethyl ester;

Indole-1-carboxylic acid ethyl ester;

4-Methyl-indole-1-carboxylic acid ethyl ester;

5-Methyl-indole-1-carboxylic acid ethyl ester;

6-Methyl-indole-1-carboxylic acid ethyl ester;

6-Chloro-indole-1-carboxylic acid ethyl ester;

3-Methyl-indole-1-carboxylic acid ethyl ester;

7-Methyl-indole-1-carboxylic acid ethyl ester;

4-Chloro-indole-1-carboxylic acid ethyl ester;

7-Chloro-indole-1-carboxylic acid ethyl ester;

Indole-1-carboxylic acid 1,1,1-trichloroethyl ester;

7-Hydroxy-indole-1-carboxylic acid ethyl ester;

6-Hydroxy-indole-1-carboxylic acid ethyl ester;

5-Hydroxy-indole-1-carboxylic acid ethyl ester;

4-Hydroxy-indole-1-carboxylic acid ethyl ester;

7-Methoxy-indole-1-carboxylic acid ethyl ester;

6-Methoxy-indole-1-carboxylic acid ethyl ester;

5-Methoxy-indole-1-carboxylic acid ethyl ester;

4-Methoxy-indole-1-carboxylic acid ethyl ester; and

5-Chloro-indole-1-carboxylic acid ethyl ester.

A preferred embodiment of the present invention relates to compounds of formulas (I), (Ia), (Ib), (II), (IIa), (IIb), or (IV) selected from the group consisting of:

1-Naphthoic acid methyl ester;

1-Naphthoic acid ethyl ester;

1-Naphthoic acid isopropyl ester;

1-Naphthoic acid propyl ester;

1-Naphthoic acid 2-hydroxyethyl ester;

1-Naphthoic acid ethylamide;

1-Naphthoic acid pentyl ester;

1-Naphthoic acid 2-propenyl ester;

1-Naphthoic acid 2-propynyl ester;

1-Naphthoic acid secbutyl ester;

1-Naphthoic acid cyclopropylmethyl ester;

1-Naphthoic acid cyclopentyl ester;

1-Naphthoic acid cyclohexyl ester;

1-Naphthoic acid-2-methoxyethyl ester; and

1-Naphthoic acid-2-methylsulfanyl ester.

A more preferred embodiment of the present invention relates to compounds of formulas (I), (Ia), (Ib), (II), (IIa), (IIb), or (IV) selected from the group consisting of:

1-Naphthoic acid methyl ester;

1-Naphthoic acid ethyl ester;

1-Naphthoic acid isopropyl ester;

1-Naphthoic acid propyl ester;

1-Naphthoic acid 2-hydroxyethyl ester;

1-Naphthoic acid ethylamide;

1-Naphthoic acid pentyl ester;

1-Naphthoic acid 2-propenyl ester;

1-Naphthoic acid 2-propynyl ester;

1-Naphthoic acid secbutyl ester;

1-Naphthoic acid cyclopropylmethyl ester;

1-Naphthoic acid cyclopentyl ester; and

1-Naphthoic acid cyclohexyl ester.

An even more preferred embodiment of the present invention relates to compounds of formulas (I), (Ia), (Ib), (II), (IIa), (IIb), or (IV) selected from the group consisting of:

1-Naphthoic acid methyl ester;

1-Naphthoic acid ethyl ester;

1-Naphthoic acid isopropyl ester;

1-Naphthoic acid propyl ester; and

1-Naphthoic acid 2-hydroxyethyl ester.

A preferred embodiment of the present invention relates to compounds of formulas (I), (Ia), (Ib), (II), (IIa), (IIc), or (V) selected from the group consisting of:

Indole-1-carboxylic acid ethyl ester;

4-Methyl-indole-1-carboxylic acid ethyl ester;

5-Methyl-indole-1-carboxylic acid ethyl ester;

6-Methyl-indole-1-carboxylic acid ethyl ester;

6-Chloro-indole-1-carboxylic acid ethyl ester;

3-Methyl-indole-1-carboxylic acid ethyl ester;

7-Methyl-indole-1-carboxylic acid ethyl ester;

4-Chloro-indole-1-carboxylic acid ethyl ester;

7-Chloro-indole-1-carboxylic acid ethyl ester;

Indole-1-carboxylic acid 1,1,1-trichloroethyl ester;

7-Hydroxy-indole-1-carboxylic acid ethyl ester;

6-Hydroxy-indole-1-carboxylic acid ethyl ester;

5-Hydroxy-indole-1-carboxylic acid ethyl ester;

4-Hydroxy-indole-1-carboxylic acid ethyl ester;

7-Methoxy-indole-1-carboxylic acid ethyl ester;

6-Methoxy-indole-1-carboxylic acid ethyl ester;

5-Methoxy-indole-1-carboxylic acid ethyl ester;

4-Methoxy-indole-1-carboxylic acid ethyl ester;

5-Chloro-indole-1-carboxylic acid ethyl ester;

Indole-1-carboxylic acid ethyl amide; and

Indole-1-carbothioic acid ethyl amide.

A more preferred embodiment of the present invention relates to compounds of formulas (I), (Ia), (Ib), (II), (IIa), (IIc), or (V) selected from the group consisting of:

Indole-1-carboxylic acid ethyl ester;

4-Methyl-indole-1-carboxylic acid ethyl ester;

5-Methyl-indole-1-carboxylic acid ethyl ester;

6-Methyl-indole-1-carboxylic acid ethyl ester;

6-Chloro-indole-1-carboxylic acid ethyl ester;

3-Methyl-indole-1-carboxylic acid ethyl ester;

7-Methyl-indole-1-carboxylic acid ethyl ester;

4-Chloro-indole-1-carboxylic acid ethyl ester; and

7-Chloro-indole-1-carboxylic acid ethyl ester.

An even more preferred embodiment of the present invention relates to a compound of formulas (I), (Ia), (Ib), (II), (IIa), (IIc), or (V):

Indole-1-carboxylic acid ethyl ester.

A preferred embodiment of the present invention relates to compounds of formulas (I), (Ia), (Ib), (III), (IIIa), or (VI) selected from the group consisting of:

1-Propoxy-naphthalene;

Methyl-[1]naphthyl sulphide;

Ethyl-[1]naphthyl sulphide;

1-Propyl-sulfanyl-1-naphthalene;

1-Butyl-sulfanyl-1-naphthalene;

3-Ethenyl-sulfanyl-1-naphthalene;

3-Ethynyl-sulfanyl-1-naphthalene;

2-Hydroxyethyl-1-naphthyl sulphide;

3-Hydroxypropyl-1-naphthyl sulphide;

3-Dimethylaminopropyl-1-naphthyl sulphide;

Methyl β-(1-naphthylthio)-propionate;

β-(1-Naphthylthio)-propionic acid;

(Naphthalen-1-ylsulfanyl)-acetonitrile;

2-Dimethylaminoethyl-1-naphthyl sulphide;

Isopropyl-1-naphthyl sulphide;

sec-Butyl-1-naphthyl sulfide;

isobutyl-1-naphthyl sulfide;

Cyclohexyl-1-naphthyl sulphide;

Cyclopentyl-1-naphthyl sulphide; and

(2-Methoxy-ethylsulfanyl)-1-naphthalene.

A more preferred embodiment of the present invention relates to compounds of formulas (I), (Ia), (Ib), (III), (IIIa), or (VI) selected from the group consisting of:

1-Propoxy-naphthalene;

Methyl-[1]naphthyl sulphide;

Ethyl-[1]naphthyl sulphide;

1-Propyl-sulfanyl-1-naphthalene;

1-Butyl-sulfanyl-1-naphthalene;

3-Ethenyl-sulfanyl-1-naphthalene;

3-Ethynyl-sulfanyl-1-naphthalene;

2-Hydroxyethyl-1-naphthyl sulphide;

3-Hydroxypropyl-1-naphthyl sulphide;

3-Dimethylaminopropyl-1-naphthyl sulphide;

Methyl β-(1-naphthylthio)-propionate;

β-(1-Naphthylthio)-propionic acid; and

(Naphthalen-1-ylsulfanyl)-acetonitrile.

An even more preferred embodiment of the present invention relates to compounds of formulas (I), (Ia), (Ib), (III), (IIIa), or (VI) selected from the group consisting of:

Ethyl-[1]naphthyl sulphide;

1-Propyl-sulfanyl-1-naphthalene;

3-Ethenyl-sulfanyl-1-naphthalene;

3-Ethynyl-sulfanyl-1-naphthalene; and

(Naphthalen-1-ylsulfanyl)-acetonitrile.

A preferred embodiment of the present invention relates to compounds of formulas (I), (Ia), or (III) selected from the group consisting of:

7-Ethylsulfanyl-1H-indole;

7-Ethylsulfanyl-benzofuran;

4-Ethylsulfanyl-1H-indole; and

4-Ethylsulfanyl-benzofuran.

Activity Profile

The inventors have surprisingly found that compounds of formula (I) have an improved activity profile compared to previously reported compounds in WO 2007/076875. The compounds of the present invention have for example improved EC₅₀ values.

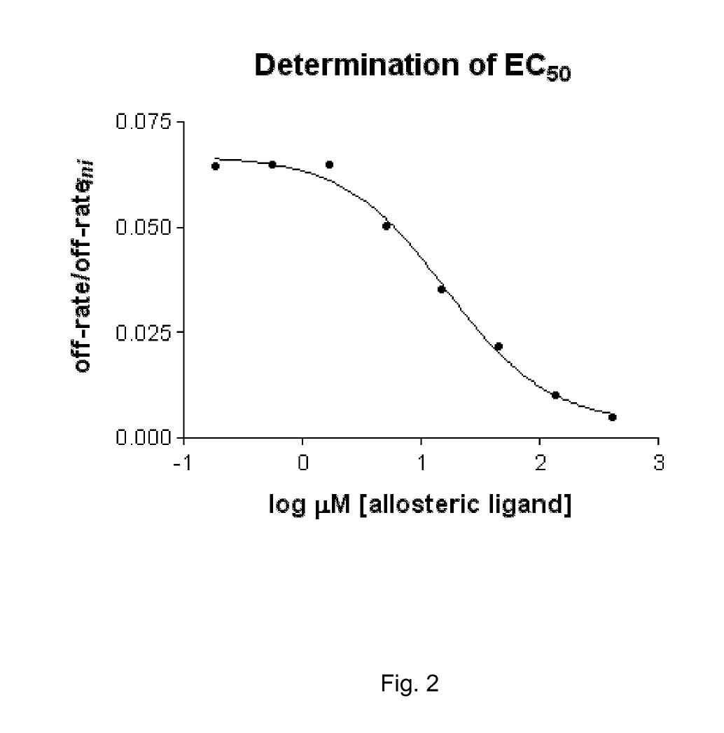

Preferred embodiments of the present invention are compounds of formula (I) which have an EC₅₀ at or below 50 μM, preferably at or below 20 μM; more preferably at or below 15 μM, even more preferably at or below 10 μM, and yet even more preferably at or below 5 μM. The EC₅₀ as used herein is defined as the concentration of allosteric compound which induces a 50% attenuation of the off-rate of the radioligand, compared to the off-rate of radioligand in the absence of allosteric compound, and may be measured by for example the dissociation assay described in example 40 and illustrated in FIG. 2.

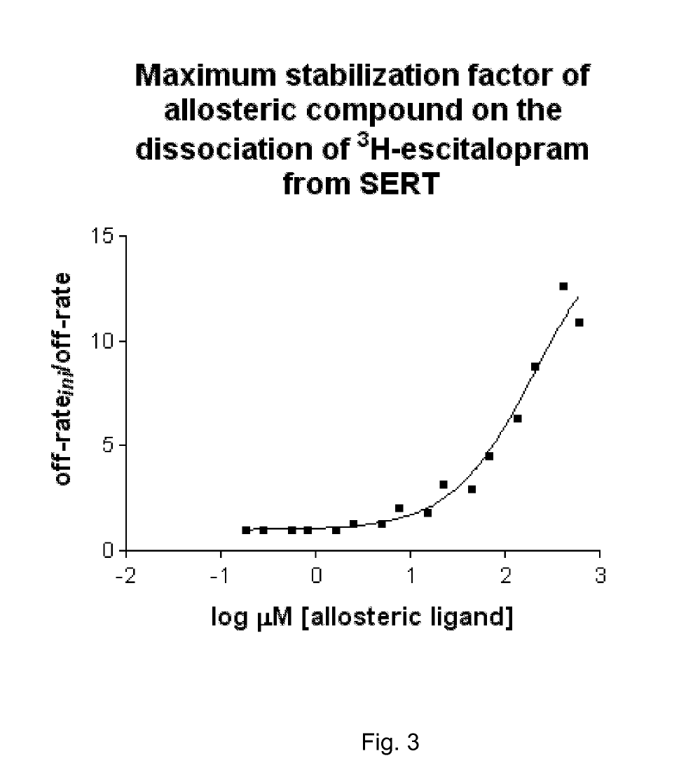

Preferred embodiments of the present invention are compounds of formula (I) which have a maximum stabilization factor above 7; preferably above 8, more preferably above 10, and even more preferably above 12. The term “maximum stabilization factor” as used herein is defined as the estimated upper plateau of a sigmoidal response curve, such as for example a sigmoidal response curve depicted on FIG. 3, and described in example 40. The maximum stabilization factor describes how many folds the off-rate of bound radio ligand from SERT can be attenuated in the presence of increasing concentrations of allosteric compound in the dissociation buffer, and may be measured by for example the dissociation assay described in example 40 and illustrated in FIG. 3.

More preferred embodiments of the present invention are compounds of formula (I) which have an EC₅₀ at or below 20 μM together with a Maximum stabilization factor above 7. Even more preferred compounds of formula (I) have an EC₅₀ at or below 15 μM together with a Maximum stabilization factor above 8. Yet even more preferred compounds of formula (I) have an EC₅₀ at or below 10 μM together with a Maximum stabilization factor above 10. Most preferred compounds of formula (I) have an EC₅₀ at or below 5 μM together with a Maximum stabilization factor above 12.

The present inventors have found that it is preferred that compounds of formula (I) have a limited size in the R¹ or R² part of the compounds. For example by limiting the chain length of alkyl chains or limiting the size of ring systems increasingly preferred properties, i.e. EC₅₀ values and maximum stabilization, are obtained. For example compounds having a C₁₋₄ alkyl chain as the R¹ or R² moiety, which ever is appropriate, have an improved activity profile compared to compounds having a C₅₋₈ alkyl chain. The same applies where the R¹ or R² moiety is e.g. a cycloalkyl moiety, i.e. a C₃₋₅ cycloalkyl moiety gives an improved activity profile compared to compounds having a C₆₋₈ cycloalkyl moiety.

Furthermore, the inventors have surprisingly found that the compounds of the present invention demonstrate a synergistic effect when administered together with one or more active substances, such as when administered together with one or more psychiatric medications or one or more antidepressants. Preferably the compounds of the present invention is administered in combination with one or more active substances selected from the group consisting of selective serotonin reuptake inhibitors (SSRIs), tricyclic antidepressiva (TCAs), serotonin-norepinephrine reuptake inhibitors (SNRIs), and Serotonin-norepinephrine-dopamine-reuptake-inhibitors (SNDRIs). Most preferred the one or more active substances are selective serotonin reuptake inhibitors (SSRIs) selected from the group consisting of citalopram, escitalopram, fluoxetine, paroxetine, sertraline, fluvoxamine, venlafaxine, duloxetine, zimelidine, and dapoxetine.

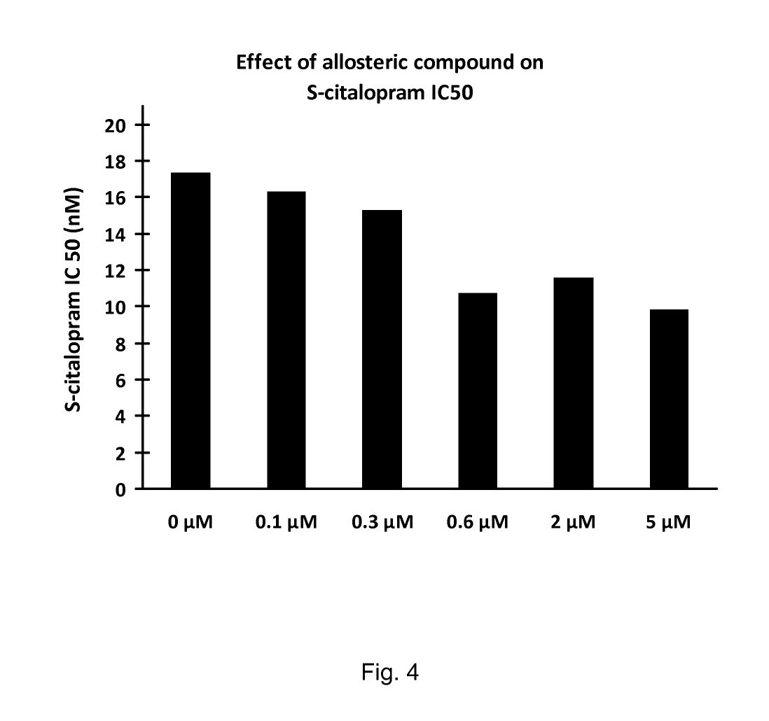

Preferred embodiments of the present invention relates to compounds, which demonstrate a synergistic effect in form of inducing a faster onset of action of the psychiatric medication, while other preferred embodiments relate to compounds, which show a synergistic effect in form of an increased efficacy, such as an add-on inhibitory effect of the uptake of serotonin as shown in FIG. 4.

CNS Disorders

The present invention relates to compounds of formula (I), for use as a medicament, and especially for treating CNS disorders, such as e.g. depression. The present invention further relates to use of compounds of formula (I) for the preparation of a medicament for the treatment of CNS disorders.

In one embodiment of the invention the CNS disorders to be treated by compounds of the present invention is selected from the group consisting of depression, panic disorder, anxiety, obsessive-compulsive disorder (OCD), generalized anxiety disorder (GAD), social phobia, bulimia nervosa, anorexia nervosa, post-traumatic stress disorder (PTSD), and neuropathic pain.

In a preferred embodiment of the invention the CNS disorder is selected from the group consisting of depression, panic disorder, anxiety, and obsessive-compulsive disorder. In a more preferred embodiment of the invention the CNS disorder is selected from the group consisting of depression and anxiety. In an even more preferred embodiment of the invention the CNS disorder is depression.

Pharmaceutical Compositions

A compound of this invention may be administered alone or in combination with pharmaceutically acceptable carriers, diluents, or excipients in either single or multiple doses. Suitable pharmaceutical acceptable carriers, diluents and excipients include inert solid diluents or fillers, sterile aqueous solutions and various organic solvents. The pharmaceutical compositions formed by combining a compound of formula (I), or a pharmaceutically acceptable salt, solvate or prodrug thereof, with pharmaceutical acceptable carriers, diluents or excipients can be readily administered in a variety of dosage forms such as tablets, powders, lozenges, syrups, suppositories, injectable solutions and the like. In powders, the carrier is a finely divided solid such as talc or starch which is in a mixture with the finely divided active component. In tablets, the active component is mixed with the carrier having the necessary binding properties in suitable proportions and compacted in the shape and size desired.

Suitable carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. A preferred form for oral use are capsules, which include the formulation of the active compound with encapsulating material as a carrier providing a capsule in which the active component with or without other carriers, is surrounded by a carrier, which is thus in association with it. Thus, for purposes of oral administration, tablets containing various excipients such as sodium citrate, calcium carbonate and calcium phosphate may be employed along with various disintegrants such as starch, methylcellulose, alginic acid and certain complex silicates, together with binding agents such as polyvinylpyrrolidone, sucrose, gelatin and acacia. Additionally, lubricating agents such as magnesium stearate, sodium lauryl sulfate and talc are often useful for tabletting purposes. Solid compositions of a similar type may also be employed as fillers in soft and hard filled gelatin capsules. Preferred materials for this include lactose or milk sugar and high molecular weight polyethylene glycols. When aqueous suspensions or elixirs are desired for oral administration, the essential active ingredient therein may be combined with various sweetening or flavoring agents, coloring matter or dyes and, if desired, emulsifying or suspending agents, together with diluents such as water, ethanol, propylene glycol, glycerin and combinations thereof.

For preparing suppositories, a low melting wax, such as a mixture of fatty acid glycerides or cocoa butter, is first melted and the active component is dispersed homogeneously therein, as by stirring. The molten homogenous mixture is then poured into convenient size molds, allowed to cool, and thereby to solidify.

For parenteral administration, solutions containing a compound of this invention or a pharmaceutically acceptable salt, solvate or prodrug thereof in sesame or peanut oil, aqueous propylene glycol, or in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. The oily solutions are suitable for intra-articular, intra-muscular and subcutaneous injection purposes. The preparation of all these solutions under sterile conditions is readily accomplished by standard pharmaceutical techniques well known to those skilled in the art.

It is preferred to use parenteral administration for compounds of the invention, wherein the active part of the molecule contains acid labile groups, such as e.g. ester groups. By using parenteral administration the acidic environment of the stomach is avoided together with the first-pass metabolism. When compounds of the invention is formulated as a prodrug, which relies on the first-pass metabolism for releasing the active part of the molecule, oral administration is preferred instead (or another appropriate administration form which result in a first-pass metabolism).

A compound of formula (I) or a pharmaceutically acceptable salt thereof can be administered orally, transdermally (e.g., through the use of a patch), parenterally (e.g. intravenously), rectally, or topically. In general, the daily dosage for treating a CNS disorder will generally range from about 0.0001 to about 50.0 mg/kg body weight of the patient to be treated, preferably from about 0.0001 to about 40 mg/kg body weight of the patient to be treated, such as e.g., from about 0.0002 to about 30 mg/kg, from about 0.001 to about 20 mg/kg, from about 0.0015 to about 15 mg/kg, from about 0.01 to about 10 mg/kg, from about 0.1 to about 10 mg/kg, from about 0.5 to about 20 mg/kg, and from about 0.5 to about 20 mg/kg. As an example, a compound of the formula (I) or a pharmaceutically acceptable salt, solvate or prodrug thereof can be administered for treatment of a CNS disorder to an adult human of average weight (about 70 kg) in a dose ranging from about 0.01 mg up to about 2000 mg per day, preferably from about 0.1 to about 1000 mg per day, such as e.g., from about 0.1 to about 500 mg per day, and from about 0.1 to about 100 mg per day, or such as e.g., from about 1 to about 1000 mg per day, from about 10 to about 1000 mg per day, from about 100 to about 1000 mg per day, from about 200 to about 1000 mg per day, and from about 500 to about 1000 mg per day, in single or divided (i.e., multiple) portions.

In general, the therapeutically-effective compounds of this invention (i.e. compounds of formula (I)) are present in pharmaceutical compositions at concentration levels ranging from 5% to 95% by weight, preferably from 10% to 95% by weight, such as e.g., from 20% to 95% by weight, from 30% to 95% by weight, from 40% to 95% by weight, more preferably from 50% to 95% by weight, such as e.g., from 60% to 95% by weight, and from 70% to 95% by weight.

Variations based on the aforementioned dosage ranges may be made by a physician of ordinary skill taking into account known considerations such as the weight, age, and condition of the person being treated, the severity of the affliction, and the particular route of administration chosen.

The pharmaceutical preparations of the invention are preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compounds of the invention may also be formulated in a pharmaceutical composition comprising one or more further active substances alone, or in combination with pharmaceutically acceptable carriers, diluents, or excipients in either single or multiple doses. The suitable pharmaceutical acceptable carriers, diluents and excipients are as described herein above, and the one or more further active substances may be any active substances, or preferably an active substance as described in the section “combination treatment” herein below.

Combination Treatment

The present invention furthermore relates to a combination treatment of a CNS disorder, wherein a compound of the present invention is administered in combination with one or more further active substance, preferably a psychiatric medication, such as e.g., antidepressants, stimulants, antipsychotics, mood stabilizers, anxiolytics, or depressants, more preferably one or more anti-depressants. The psychiatric medication may preferably be selected from the group consisting of Selective serotonin reuptake inhibitors (SSRIs), Tricyclic antidepressants (TCAs), serotonin-norepinephrine reuptake inhibitors (SNRIs), Noradrenergic and specific serotonergic antidepressants (NASSAs), Norepinephrine reuptake inhibitors (NRIs), Dopamine Reuptake Inhibitors (DARIs), Norepinephrine-dopamine reuptake inhibitors (NDRIs), and Serotonin-norepinephrine-dopamine-reuptake-inhibitors (SNDRIs); even more preferably one or more psychiatric medications may be selected from the group consisting of SSRIs, TCAs, SNRIs, NRIs and SNDRIs; yet even more preferably SSRIs.

In one embodiment of the present invention the compounds of formula (I) is administered in combination with one or more further active substances for the treatment of a CNS disorder, also denoted psychiatric medication. In a preferred embodiment of the invention the one or more further active substances is one or more antidepressants.

Examples of anti-depressants that can be combined with one or more compounds of formula (I), or their pharmaceutically acceptable salts, solvates or prodrugs, in a combination treatment according to the present invention, include, but are not limited to, SSRI, TCA, SNRI, NDRI and SNDRI. In a preferred embodiment of the invention the antidepressant is selected from the group consisting of SSRIs, TCAs, SNDRIs and SNRIs.

Examples of SSRIs that can be combined with one or more compounds of formula (I), or their pharmaceutically acceptable salts, solvates or prodrugs, in a method for treatment, a combination treatment or a pharmaceutical composition include, but are not limited to, citalopram, escitalopram, fluoxetine, paroxetine, sertraline, fluvoxamine, venlafaxine, duloxetine, zimelidine, and dapoxetine. In a preferred embodiment of the present invention the SSRIs are selected from the group consisting of citalopram, escitalopram, fluoxetine, paroxetine, sertraline, fluvoxamine, venlafaxine, and duloxetine. Other SSRIs may be combined or administered in combination with a compound of formula (I) or a pharmaceutically acceptable salt, solvate or prodrug thereof.

Examples of TCAs that can be combined with one or more compounds of formula (I), or their pharmaceutically acceptable salts, solvates or prodrugs, include, but are not limited to, imipramine, amitrypline, butriptyline, amoxapine, clomipramine, desipramine, dosulepin, doxepin, iprindole, lofepramine, nortriptyline, opipramol, protriptyline, and trimipramine.

Examples of SNRIs that can be combined with one or more compounds of formula (I), or their pharmaceutically acceptable salts, solvates or prodrugs, include, but are not limited to, venlafaxine and duloxetine. An example of a NASSA that can be combined with one or more compounds of formula (I), or their pharmaceutically acceptable salts, solvates or prodrugs, includes, but is not limited to, mirtazapine. Examples of NRIs that can be combined with one or more compounds of formula (I), or their pharmaceutically acceptable salts, solvates or prodrugs, include, but are not limited to, Atomoxetine, Reboxetine, Viloxazine, Maprotiline, Nortriptyline, Bupropion and Radafaxine.

Examples of NDRIs that can be combined with one or more compounds of formula (I), or their pharmaceutically acceptable salts, solvates or prodrugs, include, but are not limited to, Bupropion and Nomifensine.

Serotonin-norepinephrine-dopamine-reuptake-inhibitors (SNDRIs) are also termed Triple reuptake inhibitors, or TRIs, as these compounds block the uptake of serotonin, norepinephrine and dopamine. Examples of SNDRIs that can be combined with one or more compounds of formula (I), or their pharmaceutically acceptable salts, solvates or prodrugs, include, but are not limited to, tesofensine, brasofensine, diclofensine, and NS2359.

The compound of formula (I) and the one or more further active substance, such as e.g., a SSRI, can be administered to the mammal at the same time and/or at different times. Moreover, they may be administered together in a single pharmaceutical composition or in separate pharmaceutical compositions.

The therapeutically effective amount of a further active substance can generally be determined by a person skilled in the art. A proposed effective daily dosage range for a further active substance, such as preferably a SSRI, in combination with a compound of formula (I) is from about 0.01 to about 500 mg/kg body weight. The effective daily amount of the compound of formula (I) generally will be between about 0.0001 to about 10 mg/kg body weight. In some embodiments of the invention, the amount of the further active substance, such as preferably a SSRI, and/or the amount of compound of formula (I), in the combination may be less than would be required on an individual basis to achieve the same desired effect in treating depression or anxiety, due to the observed synergistic effect of combining compounds of formula (I) with e.g. a SSRI.

Method of Treatment

In a further aspect the present invention relates to a method of treating diseases in a subject, said method comprises administering to said subject a therapeutically effective amount of a compound of formula (I), or subformulas thereof described herein, or pharmaceutically acceptable salts, solvates or prodrugs thereof, as defined herein, to a subject in need of such treatment. The disease may be any disease or disorder as mentioned herein, such as for example mentioned in the section “CNS disorders”, and the compound may be administered alone or in a pharmaceutical composition, such as for example mentioned in the section “Pharmaceutical compositions”.

In a preferred embodiment of this aspect of the invention the method is a method of treating a CNS disorder in a subject, said method comprises administering to said subject a therapeutically effective amount of a compound of formula (I), or subformulas thereof described herein, or pharmaceutically acceptable salts, solvates or prodrugs thereof, as defined herein, to a subject in need of such treatment. The CNS disorder may be any CNS disorder as described herein above. Preferably the CNS disorder is depression.

In one embodiment of the method according to the invention, the compound of formula (I), or subformulas thereof described herein, or pharmaceutically acceptable salts, solvates or prodrugs thereof, as defined herein, is administered in combination with one or more further active substances. The active substances may be any active substances, and preferably an active substance as described herein above in the section “combination treatment”. More preferably the one or more additional active substances are selected from the group consisting of SSRI, TCA, SNDRI or SNRI. Even more preferably the one or more further active substances are SSRIs.

Synthesis

Compounds of formula (I), as defined herein, and pharmaceutically acceptable salts, solvates and prodrugs thereof, can be prepared according to the following reaction Schemes and discussion. In these Schemes ring system A+B is exemplified by a naphthyl moiety, however, other ring moieties as described herein, may likewise be employed. Unless otherwise indicated A, B, Y, X₁, X₂, X₃, Z, n, R¹, R², R³, and R⁴ are as defined above. Isolation and purification of the products is accomplished by standard procedures which are known to a chemist of ordinary skill.

As used herein, the expression “reaction inert solvent” refers to a solvent system in which the components do not interact with starting materials, reagents, or intermediates of products in a manner which adversely affects the yield of the desired product.

During any of the following synthetic sequences it may be necessary and/or desirable to protect sensitive or reactive groups on any of the molecules concerned. This may be achieved by means of conventional protecting groups, such as those described in T. W. Greene, Protective Groups in Organic Chemistry, John Wiley & Sons, 1981; and T. W. Greene and P. G. M. Wuts, Protective Groups in Organic Chemistry, John Wiley & Sons, 1991.

Compounds of formula (I) having formula (II), wherein L₁ is —O—, and R¹ is alkyl, cycloalkyl, alkenyl, cycloalkenyl, alkynyl, heterocyclyl, aryl, or heteroaryl, or any one of these attached to L₁ via an alkyl group, may be synthesized by any well known procedure, for example by alkylation of the corresponding carboxylic acid with a halide, like a bromide or iodide, and a base like cesium carbonate as described by Lee et al. (Lee, J. C., Oh, Y. S., Cho, S. H., Lee, J. I., Org. Prep. Proc. Int. 1996, 28, 480-483), in a suitable reaction inert solvent, such as e.g. acetonitrile or dimethylformamide. Scheme 1 illustrates a suitable method for preparing these compounds.

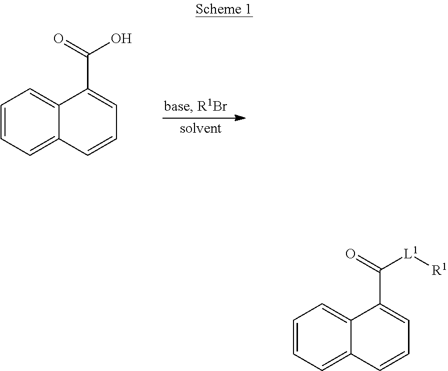

Compounds of formula (I) having formula (IIc), wherein Z is S, and L₁ is —NH— or —NR³—, i.e. thioureas, may be prepared by reacting an indole or indoline with the appropriate isothiocyanate.

Compounds of formula (I) having formula (IIc), wherein Z is S, and L₁ is —O—, i.e. thiocarbamates, may be prepared by e.g. reacting an indole or indoline with the appropriate alkyl, alkenyl, alkynyl, aryl, heterocyclyl etc. chlorothionoformate.

Compounds of formula (I) having formula (II), wherein L₁ is —NH—, —NR³—, or —C— and R¹ is alkyl, cycloalkyl, alkenyl, cycloalkenyl, alkynyl, heterocyclyl, aryl, or heteroaryl, or any one of these attached to L₁ via an alkyl group, may be synthesized by any well known procedure, for example by reaction of a carboxylic acid chloride with an amine or organometallic reagent in any suitable reaction inert solvent, such as e.g. acetonitrile or tetrahydrofuran. Scheme 2 illustrates a suitable method for preparing these compounds.

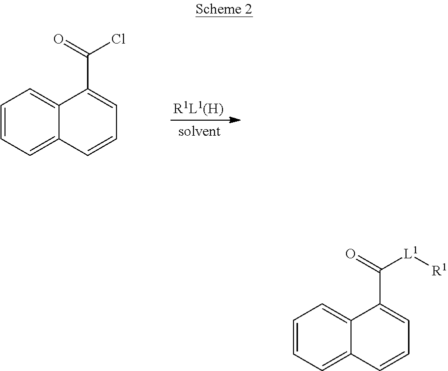

Compounds of formula (I) having formula (III), wherein L₂ is —S— or —O— and R² is alkyl, cycloalkyl, alkenyl, cycloalkenyl, alkynyl, heterocyclyl, aryl, or heteroaryl, or any one of these attached to L₂ via an alkyl group, may be synthesized by any well known procedure, for example by alkylation with a halide, like a bromide or iodide, and under the presence of a suitable base, like potassium carbonate or triethylamine, in any reaction inert solvent, such as e.g. acetonitrile or dimethylformamide. Scheme 3 illustrates a suitable method for preparing these compounds.

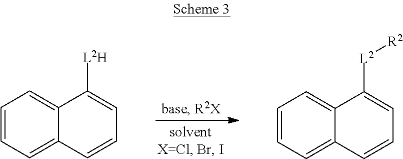

Compounds of formula (I) having formula (III), wherein L₂ is —NH— or —NR⁴—, may be synthesized by any well known procedure, for example by alkylation of 1-aminonaphthalene with an electrophile like an alkyl halide, e.g., a bromide or chloride, under the presence of a suitable base, in a reaction inert solvent, such as e.g. ethanol, and under heating conditions. Scheme 4 illustrates a suitable method for preparing these compounds.

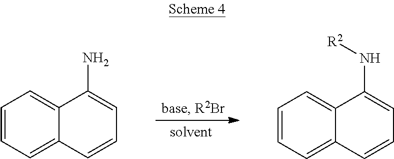

Compounds of formula (I) having formula (V), wherein L₁ is —O—, —NH—, NR³—, or —C— and R¹ is alkyl, cycloalkyl, alkenyl, cycloalkenyl, alkynyl, heterocyclyl, aryl, or heteroaryl, or any one of these attached to L₁ via an alkyl group, may be synthesized by any well known procedure, for example by acylation with chloroformates in the presence of a suitable base, such as e.g. triethylamine or sodium hydride, in a suitable reaction inert solvent, such as e.g. a solvent like dichloromethane or tetrahydrofuran. Scheme 5 illustrates a suitable method for preparing these compounds.

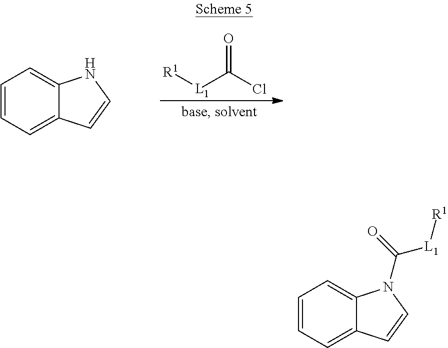

All patent and non-patent references cited in the application, or in the present application, are also hereby incorporated by reference in their entirety.

The following Examples illustrate the present invention. It is to be understood, however, that the invention, as fully described herein and as recited in the claims, is not intended to be limited by the details of the following Examples. Those having skill in the art will recognize that the starting materials may be varied and additional steps employed to produce compounds encompassed by the present invention, as demonstrated by the following examples.

### EXAMPLES

In the examples below commercial available starting materials and reagents were used without further purification. Solvents were dried according to standard procedures. Columns for flash chromatography were packed with silica gel (60 Å). TLC plates (Kieselgel 60 F₂₅₄) were visualized by UV-light or the use of a “Ce-Mol” solution (Ce(IV)sulfate (10 g) and ammonium molybdate (15 g) dissolved in 10% H₂SO₄ (1 L)) and heated until colored spots appeared. ¹H and ¹³C NMR experiments were recorded on a Varian Mercury 400 NMR instrument. Mass spectral analyses were carried out as electrospray experiments on a Micromass LC-TOF instrument.

### Example 1

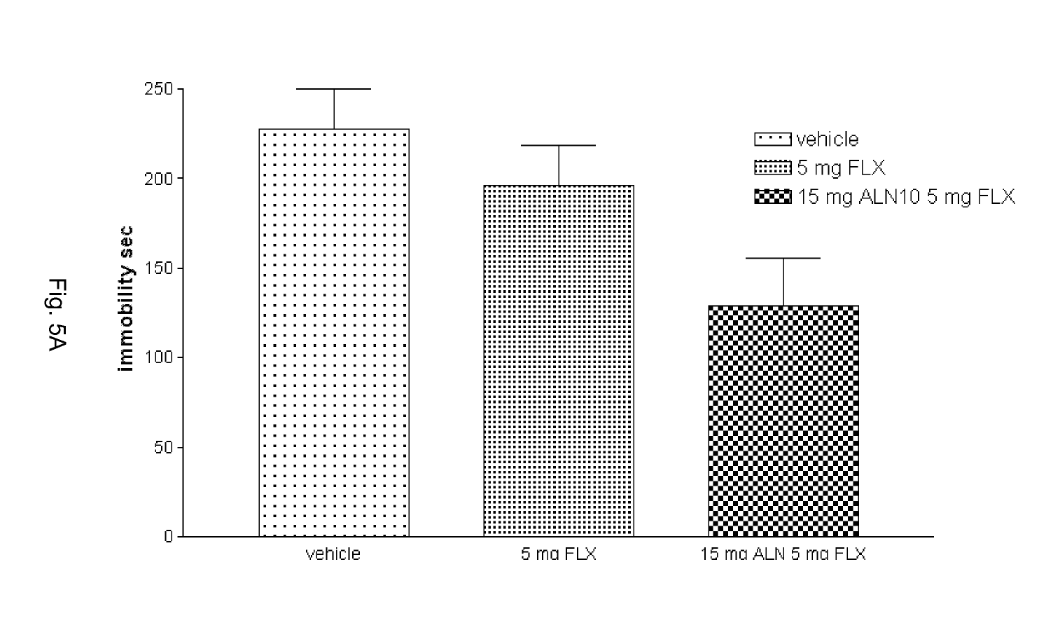

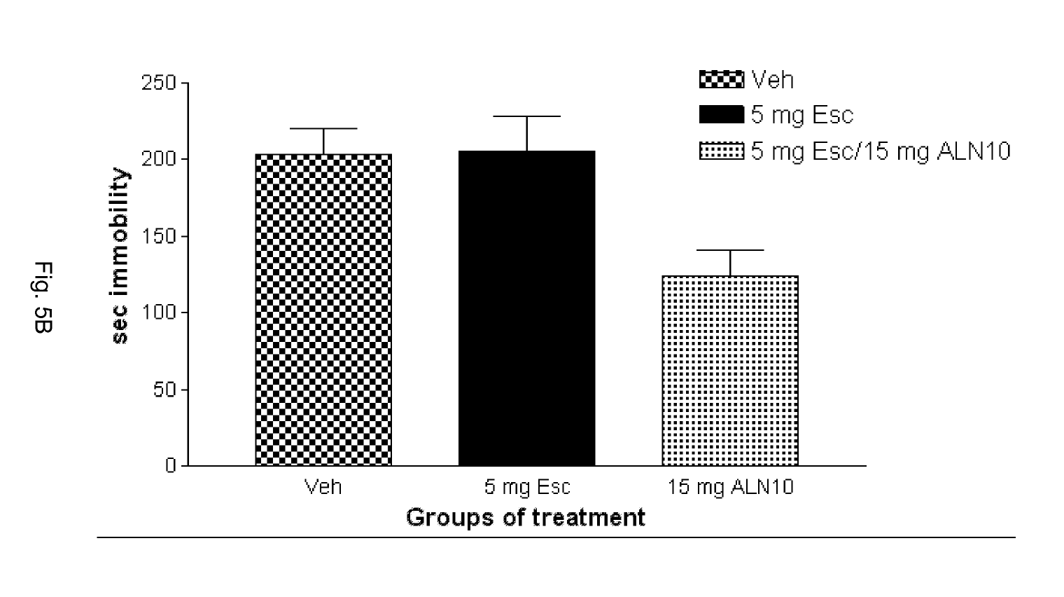

### 1-Naphthoic acid ethyl ester (also Denoted ALN10 in FIGS. 5A and 5B)

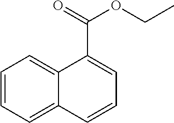

Cesium carbonate (1.99 g, 6.09 mmol) and ethyl iodide (1.62 mL, 20.3 mmol) were added at ambient temperature to a stirred solution of 1-naphthoic acid (700 mg, 4.06 mmol) in dry acetonitrile (70 mL). The reaction mixture was heated to reflux under an atmosphere of N₂ for 90 min. before it was cooled to ambient temperature and filtered. The filtrate was concentrated under reduced pressure and re-dissolved in CH₂Cl₂ (175 mL). The organic phase was then washed with an aqueous solution of NaHCO₃ (20%, 3*50 mL) and brine before dried over anhydrous Na₂SO₄, filtered and concentrated under reduced pressure. The remaining crude material was purified by column chromatography on silica (AcOEt/petrolether 1:9) to give 1-Naphthoic acid ethyl ester (643 mg, 79%). ¹H-NMR data was in accordance with: Yoshino, T., Imori, S., Togo, H., Tetrahedron, 2006, 62, 1309-1317.

¹³C-NMR (CDCl₃, 100 MHz) δ_{C} 167.8, 134.0, 133.3, 131.5, 130.2, 128.7, 127.8, 127.7, 126.3, 126.0, 124.6, 61.2, 14.5. HRMS(ES+): calcd. for C₁₃H₁₂O₂Na: 223.0735; found: 223.0733.

### Example 2

### 1-Naphthoic acid methyl ester

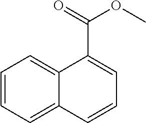

The title compound was prepared according to the procedure described in Example 1 from 1-napthoic acid and methyliodide.

¹H-NMR (CDCl₃, 400 MHz) δ_{H} 8.95 (d, 1H, J 8.4 Hz), 8.20 (d, 1H, J 7.2 Hz), 8.01 (d, 1H, J 8.0 Hz), 7.88 (d, 1H, J 8.0 Hz), 7.65-7.47 (m, 3H), 4.01 (s, 3H).

NMR data was in accordance with: Lerebours, R., Wolf, C., J. Am. Chem. Soc. 2006, 128, 13052-13053.

### Example 3

### 1-Naphthoic acid propyl ester

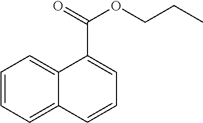

The title compound was prepared according to the procedure described in Example 1 from 1-napthoic acid and propylbromide.

¹H-NMR (CDCl₃, 400 MHz) δ_{H} 8.98 (d, 1H, J 8.4 Hz), 8.22 (d, 1H, J 7.2 Hz), 8.01 (d, 1H, J 8.0 Hz), 7.66-7.62 (m, 1H), 7.54 (t, 1H, J 7.2 Hz), 7.50 (t, 1H, J 8.0 Hz), 4.41 (t, 2H, J 6.4 Hz), 1.88 (sext, 2H, J 6.8 Hz), 1.10 (t, 3H, J 6.8 Hz).

¹³C-NMR (CDCl₃, 100 MHz) δ_{C} 167.7, 133.9, 133.3, 131.5, 130.1, 128.6, 127.7, 127.6, 126.2, 125.9, 124.6, 66.7, 22.3, 10.7.

### Example 4

### 1-Naphthoic acid isopropyl ester

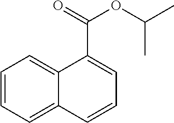

The title compound was prepared according to the procedure described in Example 1 from 1-napthoic acid and isopropylbromide

¹H-NMR (CDCl₃, 400 MHz) δ_{H} 8.95 (d, 1H, J 8.8 Hz), 8.17 (dd, 1H, J 1.2 Hz, J 7.2 Hz), 8.09 (d, 1H, J 8.0 Hz), 7.87 (d, 1H, J 8.0 Hz), 7.64-7.60 (m, 1H), 7.55-7.47 (m, 2H), 5.41 (sep, 1H, J 6.4 Hz), 1.46 (d, 6H, J 6.4 Hz).

NMR data was in accordance with: Strey, K., Voes, J., J. Chem. Res. Miniprint, 1998, 648-682.

### Example 5

### 1-Naphthoic acid pentyl ester

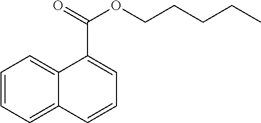

The title compound was prepared according to the procedure described in Example 1 from 1-napthoic acid and 1-bromo-pentane.

¹H-NMR (CDCl₃, 400 MHz) δ_{H} 8.92 (d, 1H, J 8.8 Hz), 8.18 (d, 1H, J 7.6 Hz), 8.02 (d, 1H, J 8.4 Hz), 7.89 (d, 1H, J 8.4 Hz), 7.64-7.49 (m, 3H), 4.44 (t, 2H, J 6.8 Hz), 1.84 (quint, 2H, J 6.8 Hz), 1.51-1.39 (m, 4H), 0.96 (t, 3H, J 6.8 Hz).

¹³C-NMR (CDCl₃, 100 MHz) δ_{C} 167.9, 134.0, 133.3, 131.5, 130.2, 128.7, 127.8, 127.7, 126.3, 126.0, 124.7, 65.4, 28.6, 28.5, 22.5, 14.2.

### Example 6

### 1-Naphthoic acid 2-hydroxyethyl ester

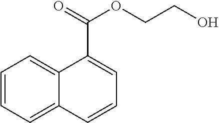

The title compound was prepared according to the procedure described in Example 1 from 1-napthoic acid and 2-bromo-ethanol.

¹H-NMR (CDCl₃, 400 MHz) δ_{H} 8.46 (d, 1H, J 8.4 Hz), 7.84 (d, 1H, J 8.0 Hz), 7.75 (d, 1H, J 8.4 Hz), 7.63-7.49 (m, 3H), 7.40-7.36 (m, 1H), 3.70 (t, 2H, J 5.6 Hz), 3.13-3.09 (m, 2H), 2.90 (b s, 1H).

NMR data was in accordance with: Sharghi, H., Sarvari, M. H., Tetrahedron, 2003, 59, 3627-3634.

### Example 7

### (±) 1,2,3,4-Tetrahydro-1-naphthoic acid ethyl ester

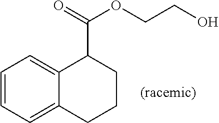

1,2,3,4-Tetrahydro-1-naphthalenecarbonitrile (408 mg, 2.6 mmol) was dissolved in 5 M KOH in isopropanol (10 mL) and heated to 100° C. overnight and then cooled to room temperature and acidified with 6 M hydrochloric acid. The solution was then extracted three times with diethylether before the combined ethereal extracts were extracted 3 times with aqueous saturated NaHCO₃. The NaHCO₃ were acidified with hydrochloric acid and extracted 3 times with CH₂Cl₂, which were dried over MgSO₄, filtered and concentrated under reduced pressure. The remaining material was dissolved in dry CH₃CN (3 mL) and ethyl bromide (0.28 mL, 3.7 mmol) and Cs₂CO₃ (608 mg, 1.87 mmol) were added and the reaction mixture heated to reflux for 2 hours. The resulting slurry was filtered and the filtrate concentrated under reduced pressure before taken up in CH₂Cl₂ (30 mL) and washed with water (30 mL). The organic layer was dried over MgSO₄, filtered, concentrated and purified by column chromatography on silica (CH₂Cl₂/pentane 1:1) to give racemic 1,2,3,4-tetrahydro-1-naphthoic acid ethyl ester (240 mg, 45%)

Racemic: ¹H-NMR (CDCl₃, 400 MHz) δ_{H} 7.19-7.09 (m, 4H), 4.18 (q, 2H, J 7.2 Hz), 3.81 (t, 1H, J 6.0 Hz), 2.88-2.72 (m, 2H), 2.18-2.09 (m, 1H), 2.05-1.94 (m, 2H), 1.80-1.72 (m, 1H), 1.27 (t, 3H, J 7.2 Hz).

¹³C-NMR (CDCl₃, 100 MHz) δ_{C} 174.9, 137.2, 133.4, 129.4, 129.2, 126.8, 125.7, 60.8, 44.9, 29.2, 26.7, 20.7, 14.3.

### Example 8

### 1-Naphthoic acid 2-propenyl ester

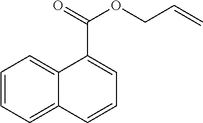

The title compound was prepared according to the procedure described in Example 1 from 1-napthoic acid and allyl bromide.

¹H-NMR (CDCl₃, 100 MHz) δ_{C} 167.4, 134.1, 133.7, 132.5, 131.6, 130.5, 128.8, 128.0, 127.3, 126.4, 126.0, 124.7, 118.7, 65.9.

NMR data was in accordance with: Merbouh, N., Wallner, F. K., Cociorva, O. M., Seeberger, P. H., Org. Lett. 2007, 9, 651-653.

### Example 9

### 1-Naphthoic acid 2-propynyl ester

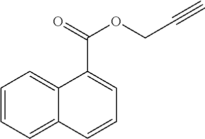

The title compound was prepared according to the procedure described in Example 1 from 1-napthoic acid and propargyl bromide.

¹H-NMR (CDCl₃, 400 MHz) δ_{H} 8.96 (d, 1H, J 8.0 Hz), 8.27 (dd, 1H, J 1.2 Hz, J 7.2 Hz), 8.05 (d, 1H, J 8.4 Hz), 7.90 (dd, 1H, J 1.6 Hz, J 8.4 Hz), 7.66-7.49 (m, 3H), 5.02 (d, 2H, J 2.4 Hz), 2.56 (t, 1H, J 2.4 Hz).

¹³C-NMR (CDCl₃, 100 MHz) δ_{C} 166.6, 134.0, 134.0, 131.5, 130.9, 128.7, 128.1, 126.4, 126.2, 125.8, 124.6, 78.0, 75.2, 52.6.

### Example 10

### 1-Naphthoic acid ethylamide

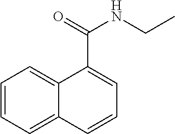

Thionyl chloride (2.5 mL, 34.8 mmol) was added in a dropwise fashion at ambient temperature with stirring to 1-naphthoic acid (500 mg, 2.9 mmol) under an atmosphere of N₂. The reaction mixture was then heated to 80° C. for 2 hours before the excess thionyl chloride was removed under reduced pressure. The residue was dissolved in THF (25 mL) and cooled on an ice-bath before ethylamine in H₂O (70%, 0.26 mL, 3.19 mmol) was added. The reaction mixture was stirred at ambient temperature overnight before it was poured onto a saturated aqueous solution of NaHCO₃ (60 mL). The mixture was then extracted with CH₂Cl₂ (3*60 mL) and the combined organic phases dried (MgSO₄), filtered, and concentrated under reduced pressure. The remaining material was purified by column chromatography (silica, AcOEt/petrolether 2:1) to give the 1-Naphthoic acid ethylamide (103 mg, 18%).

¹H-NMR (CDCl₃, 400 MHz) δ_{H} 8.29 (d, 1H, J 8.4 Hz), 7.90-7.84 (m, 2H), 7.57-7.49 (m, 3H), 7.43 (t, 1H, J 7.2 Hz), 6.03 (b s, 1H), 3.60-3.53 (m, 2H), 1.28 (t, 3H, J 7.2 Hz). ¹³C-NMR (CDCl₃, 100 MHz) δ_{C} 169.6, 134.9, 133.8, 130.5, 130.3, 128.4, 127.2, 126.5, 125.5, 124.9, 124.8, 35.1, 15.1.

### Example 11

### 1-Propyl-sulfanyl-1-naphthalene

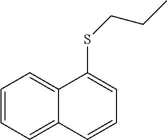

To a stirred solution of 1-naphthalenethiol (1.50 mL, 10.8 mmol) and anhydrous K₂CO₃ (2.13 g, 15.4 mmol) in dry THF (40 mL) under an atmosphere of nitrogen was added propyl bromide (1.14 mL, 13.0 mmol) at ambient temperature. The reaction mixture was stirred for 3 hours until TLC analysis (silica, pentane) indicated full consumption of starting material before it was diluted with H₂O (40 mL) and extracted with Et₂O (3*25 mL). The combined organic phases were dried over MgSO₄, filtered and concentrated under reduced pressure before purified by column chromatography on silica (eluent: pentane) to give 1-propyl-sulfanyl-1-naphthalene (1.70 g, 78%) as a colorless oil.

¹H-NMR (CDCl₃, 400 MHz) δ_{H} 8.44 (d, 1H, J 8.4 Hz), 7.85 (d, 1H, J 8.4 Hz), 7.73 (d, 1H, J 8.0 Hz), 7.59-7.50 (m, 3H), 7.42 (t, 1H, J 7.6 Hz), 2.98 (t, 2H, J 7.4 Hz), 1.71 (sext, 2H, J 7.4 Hz), 1.06 (t, 3H, J 7.4 Hz).

¹³C-NMR (CDCl₃, 100 MHz) δ_{C} 134.2, 134.0, 133.1, 128.7, 127.8, 127.0, 126.4, 126.3, 125.7, 125.2, 36.4, 22.7, 13.6.

### Example 12

### Methyl-[1]naphthyl sulphide

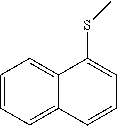

The title compound was prepared according to the procedure described in Example 11 from 1-napthalenethiol and methyliodide.

¹H-NMR (CDCl₃, 400 MHz) δ_{H} 8.33 (d, 1H, J 8.0 Hz), 7.87 (d, 1H, J 7.6 Hz), 7.70 (d, 1H, J 7.6 Hz), 7.60-7.52 (m, 2H), 7.45-7.40 (m, 2H), 2.60 (s, 3H).

NMR data was in accordance with: Schmidt, L. C., Rey, V., Penenory, A. B., Eur. J. Org. Chem. 2006, 2210-2214.

### Example 13

### Ethyl-[1]naphthyl sulphide

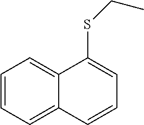

The title compound was prepared according to the procedure described in Example 11 from 1-napthalenethiol and ethyl bromide.

¹H-NMR (CDCl₃, 400 MHz) δ_{H} 8.53 (d, 1H, J 8.4 Hz), 7.91 (d, 1H, J 8.0 Hz), 7.79 (d, 1H, J 8.4 Hz), 7.65-7.56 (m, 3H), 7.50-7.45 (m, 1H), 3.07 (q, 2H, J 7.2 Hz), 1.41 (t, 3H, J 7.2 Hz).

### Example 14

### 1-Butyl-sulfanyl-1-naphthalene

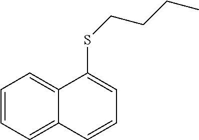

The title compound was prepared according to the procedure described in Example 11 from 1-napthalenethiol and 1-bromobutane.

¹³C-NMR (CDCl₃, 100 MHz) δ_{C} 134.4, 134.0, 132.9, 128.6, 127.3, 126.8, 126.3, 126.2, 125.6, 125.1, 33.9, 31.3, 22.1, 13.7.

### Example 15

### 2-Hydroxyethyl-1-naphthyl sulphide

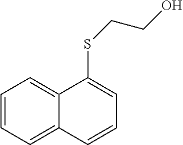

The title compound was prepared according to the procedure described in Example 11 from 1-napthalenethiol and 2-bromo-ethanol.

¹³C-NMR (CDCl₃, 100 MHz) δ_{C} 134.0, 133.1, 132.0, 129.3, 128.6, 127.8, 126.6, 126.3, 125.5, 125.0, 60.4, 37.2.

### Example 16

### 3-Ethynyl-sulfanyl-1-naphthalene

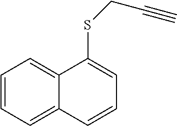

The title compound was prepared according to the procedure described in Example 11 from 1-napthalenethiol and propargyl bromide

¹H-NMR (CDCl₃, 400 MHz) δ_{H} 8.47 (d, 1H, J 8.0 Hz), 7.89 (d, 1H, J 8.0 Hz), 7.83 (d, 1H, J 8.4 Hz), 7.79 (d, 1H, J 7.6 Hz), 7.61-7.54 (m, 2H), 7.47 (t, 1H, J 8.0 Hz), 3.68 (d, 2H, J 2.0 Hz), 2.26 (t, 1H, J 2.0 Hz).

¹³C-NMR (CDCl₃, 100 MHz) δ_{C} 134.0, 133.2, 131.9, 130.2, 128.7, 128.6, 128.5, 126.8, 126.4, 125.7, 125.1, 79.8, 72.0, 23.0.

### Example 17

### 3-Ethenyl-sulfanyl-1-naphthalene

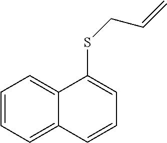

The title compound was prepared according to the procedure described in Example 11 from 1-napthalenethiol and allyl bromide.

¹H-NMR (CDCl₃, 400 MHz) δ_{H} 8.48 (d, 1H, J 7.6 Hz), 7.87 (d, 1H, J 8.0 Hz), 7.79 (d, 1H, J 8.4 Hz), 7.61-7.52 (m, 3H), 7.43 (t, 1H, 8.0 Hz), 5.99-5.89 (m, 1H), 5.12-5.05 (m, 2H), 3.65-3.61 (m, 2H).

¹³C-NMR (CDCl₃, 100 MHz) δ_{C} 134.0, 133.6, 133.3, 133.1, 129.3, 128.7, 127.6, 126.5, 126.3, 125.6, 125.2, 117.8, 37.7.

### Example 18

### 3-Hydroxypropyl-1-naphthyl sulphide

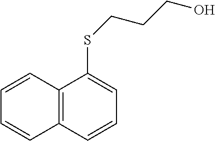

The title compound was prepared according to the procedure described in Example 11 from 1-napthalenethiol and 3-bromo-propan-1-ol.

¹H-NMR (CDCl₃, 400 MHz) δ_{H} 8.44 (d, 1H, J 8.4 Hz), 7.86 (d, 1H, J 8.0 Hz), 7.74 (d, 1H, J 8.4 Hz), 7.60-7.52 (m, 3H), 7.41 (t, 1H, J 8.0 Hz), 3.73 (t, 2H, J 6.0 Hz), 3.08 (t, 2H, J 7.2 Hz), 2.14 (s, 1H), 1.91-1.85 (m, 1H).

¹³C-NMR (CDCl₃, 100 MHz) δ_{C} 134.0, 133.5, 133.0, 128.6, 128.0, 127.2, 126.4, 126.3, 125.6, 125.0, 61.3, 31.8, 30.7.

### Example 19

### 2-Dimethylaminoethyl-1-naphthyl sulfide

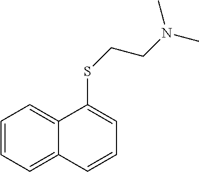

The title compound was prepared according to the procedure described in Example 11 from 1-napthalenethiol and 2-chloro-N,N-dimethylethylamine.

¹H-NMR (CDCl₃, 400 MHz) δ_{H} 8.45 (d, 1H, J 7.6 Hz), 7.83 (d, 1H, J 8.0 Hz), 7.72 (d, 1H, J 8.4 Hz), 7.60-7.48 (m, 3H), 7.41 (dd, 1H, J 7.6 Hz, J 8.4 Hz), 3.12-3.09 (m, 2H), 2.62-2.58 (m, 2H), 2.27 (s, 6H).

¹³C-NMR (CDCl₃, 100 MHz) δ_{C} 133.9, 133.8, 132.9, 128.5, 127.6, 127.1, 126.3, 126.2, 125.6, 125.1, 58.6, 45.3, 32.1.

### Example 20

### 3-Dimethylaminopropyl-1-naphthyl sulfide

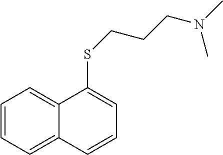

The title compound was prepared according to the procedure described in Example 11 from 1-napthalenethiol and 3-chloro-N,N-dimethylpropylamine.

¹H-NMR (CDCl₃, 400 MHz) δ_{H} 8.45 (d, 1H), 7.8 (d, 1H, J 8.4 Hz), 7.71 (d, 1H, J 8.0 Hz), 7.60-7.48 (m, 3H), 7.40 (t, 1H, J 8.0 Hz), 3.02 (t, 2H, J 7.2 Hz), 2.39 (t, 2H, J 7.2 Hz), 2.21 (s, 6H), 1.83 (quint, 2H, J 7.2 Hz).

¹³C-NMR (CDCl₃, 100 MHz) δ_{C} 133.9, 133.9, 132.9, 128.5, 127.7, 126.9, 126.3, 126.1, 125.5, 125.0, 58.5, 45.4, 32.0, 27.2.

### Example 21

### 1-Propoxy-naphthalene

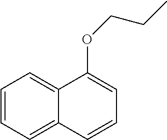

The title compound was prepared according to the procedure described in Example 11 from 1-naphthol and 1-bromopropane.

¹H-NMR (CDCl₃, 400 MHz) δ_{H} 8.47-8.44 (m, 1H), 7.92-7.89 (m, 1H), 7.60-7.44 (m, 4H), 6.88 (d, 1H, J 7.6 Hz), 4.17 (q, 2H, J 7.2 Hz), 2.05 (quint, 2H, J 7.2 Hz), 1.24 (t, 3H, J 7.2 Hz).

¹³C-NMR (CDCl₃, 100 MHz) δ_{C} 155.0, 134.7, 127.5, 126.4, 126.0, 125.9, 125.1, 122.2, 120.1, 104.7, 69.7, 22.8, 10.9.

### Example 22

### Methyl β-(1-naphthylthio)-propionate

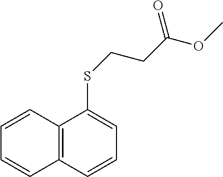

Borax was dissolved in H₂O (4.5 mL) and N₂ bobbled through the solution for 5 min. before 1-naphthalenethiol ((0.70 g, 4.4 mmol) and methyl acrylate (0.44 mL, 4.8 mmol) were added under an atmosphere of N₂ at ambient temperature. The solution was stirred for 30 min. before extracted with CH₂Cl₂ (3*10 mL). The combined organic layers were dried over MgSO₄, filtered, and concentrated under reduced pressure to give a crude oil that was purified by column chromatography on silica (eluent: CH₂Cl₂/pentane 1:3→1:1). This gave the title compound as a colorless oil (0.927 g, 86%).

¹H-NMR (CDCl₃, 400 MHz) δ_{H} 8.43 (d, 1H, J 8.0 Hz), 7.86 (d, 1H, J 8.0 Hz), 7.78 (d, 1H, J 8.0 Hz), 7.65-7.50 (m, 3H), 7.42 (t, 1H, J 8.0 Hz), 3.66 (s, 3H), 3.22 (t, 2H, J 7.2 Hz), 2.62 (t, 2H, J 7.2 Hz).

¹³C-NMR (CDCl₃, 100 MHz) δ_{C} 172.3, 134.2, 133.6, 132.3, 130.0, 128.8, 128.3, 126.8, 126.5, 125.7, 125.4, 51.9, 34.4, 29.7.

### Example 23

### β-(1-Naphthylthio)-propionic acid

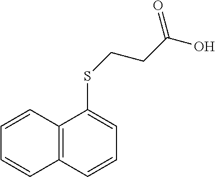

Methyl ester (Methyl β-(1-naphthylthio)-propionate) 0.61 g, 2.5 mmol) was stirred vigorously in THF (2 mL) and aqueous NaOH (1 M, 2 mL) at ambient temperature overnight. The reaction mixture was diluted with H₂O (5 mL) and washed with Et₂O (2*10 mL). The aqueous solution was then acidified with dilute HCl (aq. 1M) to pH 2 and extracted with CH₂Cl₂ (3*10 mL). The combined organic layers were dried over MgSO₄ and concentrated under reduced pressure to give β-(1-Naphthylthio)-propionic acid (0.56 g, 96%) as a colorless solid.

¹H-NMR (CDCl₃, 400 MHz) δ_{H} 8.45 (d, 1H, J 8.4 Hz), 7.86 (d, 1H, J 8.0 Hz), 7.89 (d, 1H, J 8.0 Hz), 7.66 (d, 1H, J 7.6 Hz), 7.58 (t, 1H, J 7.6 Hz), 7.53 (t, 1H, J 7.6 Hz), 7.43 (t, 1H, J 8.0 Hz), 3.20 (t, 2H, J 7.2 Hz), 2.67 (t, 2H, J 7.2 Hz).

¹³C-NMR (CDCl₃, 100 MHz) δ_{C} 178.4, 134.2, 133.5, 132.0, 130.3, 128.8, 128.4, 126.8, 126.5, 125.7, 125.3, 34.4, 29.3.

### Example 24

### Indole-1-carboxylic acid ethyl ester

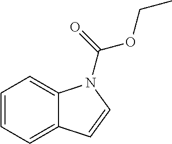

To a stirred solution of indole (0.40 g, 3.4 mmol) in dry THF (10 mL) at 0° C. was added NaH (60%, 0.27 g, 6.8 mmol). The reaction mixture was stirred for 30 min. before ethyl chloroformate (0.48 mL, 5.1 mmol) was added and the cold bath removed. After 2 hours TLC analysis indicated full consumption of indole. The reaction mixture was then diluted with H₂O (10 mL) and extracted with AcOEt (3*20 mL) before the combined organic layers were dried over MgSO₄, filtered and concentrated under reduced pressure. The remaining material was purified by column chromatography on silica (AcOEt/pentane 1:20) to give Indole-1-carboxylic acid ethyl ester (475 mg, 73%).

¹H-NMR data was in accordance with: Hiroya, K., Itoh, S., Sakamoto, T., J. Org. Chem. 2004, 69, 1126-1136.

¹³C-NMR (CDCl₃, 100 MHz) δ_{C} 151.1, 135.3, 130.6, 125.6, 124.5, 123.0, 121.0, 115.2, 108.0, 63.2, 14.5.

### Example 25

### 4-Methyl-indole-1-carboxylic acid ethyl ester

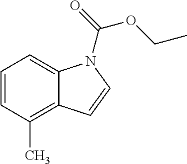

The title compound was prepared according to the procedure described in Example 24 from 4-methyl-indole and ethyl chloroformate

¹H-NMR (CDCl₃, 400 MHz) δ_{H} 8.04 (b s, 1H), 7.63 (b s, 1H), 7.28-7.22 (m, 1H), 7.08-7.06 (m, 1H), 6.66-6.64 (m, 1H), 4.55-4.48 (m, 2H), 2.55 (b s, 3H), 1.50-1.47 (m, 3H).

¹³C-NMR (CDCl₃, 100 MHz) δ_{C} 151.3, 135.1, 130.5, 130.2, 125.1, 124.6, 123.4, 112.8, 106.4, 63.2, 18.6, 14.5.

### Example 26

### 5-Methyl-indole-1-carboxylic acid ethyl ester

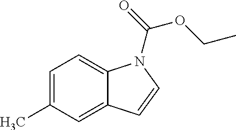

The title compound was prepared according to the procedure described in Example 24 from 5-methyl-indole and ethyl chloroformate.

¹H-NMR (CDCl₃, 400 MHz) δ_{H} 8.07 (b d, 1H), 7.60 (d, 1H, J 3.2 Hz), 7.37 (s, 1H), 7.17 (d, 1H, J 8.4 Hz), 6.54 (d, 1H, J 3.6 Hz), 4.50 (q, 2H, J 7.2 Hz), 2.47 (s, 3H), 1.48 (t, 3H, J 7.2 Hz).

¹³C-NMR (CDCl₃, 100 MHz) δ_{C} 151.2, 133.5, 132.4, 130.8, 125.9, 125.7, 121.0, 114.9, 107.8, 63.1, 21.4, 14.5.

### Example 27

### 6-Methyl-indole-1-carboxylic acid ethyl ester

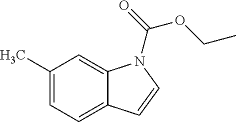

The title compound was prepared according to the procedure described in Example 24 from 6-methyl-indole and ethyl chloroformate.

¹H-NMR (CDCl₃, 400 MHz) δ_{H} 8.04 (b s, 1H), 7.50 (d, 1H, J 4.0 Hz), 7.45 (d, 1H, J 8.0 Hz), 6.55 (d, 1H, J 4.0 Hz), 4.49 (q, 2H, J 7.2 Hz), 2.51 (s, 3H), 1.48 (t, 3H, J 7.2 Hz).

¹³C-NMR (CDCl₃, 100 MHz) δ_{C} 151.2, 135.8, 134.6, 128.3, 125.0, 124.5, 120.6, 115.5, 107.9, 63.2, 22.1, 14.5.

### Example 28

### 6-Chloro-indole-1-carboxylic acid ethyl ester

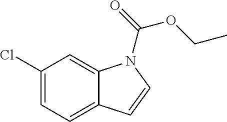

The title compound was prepared according to the procedure described in Example 24 from 6-chloro-indole and ethyl chloroformate.

¹H-NMR (CDCl₃, 400 MHz) δ_{H} 8.21 (b s, 1H), 7.59 (d, 1H, J 3.2 Hz), 7.45 (d, 1H, J 8.4 Hz), 7.21 (d, 1H, J 8.4 Hz), 6.55 (d, 1H, J 3.6 Hz), 4.50 (q, 2H, J 6.8 Hz), 1.47 (t, 3H, J 6.8 Hz).

¹³C-NMR (CDCl₃, 100 MHz) δ_{C} 150.8, 135.7, 130.5, 129.1, 126.2, 123.6, 121.7, 115.5, 107.7, 63.6, 14.5.

### Example 29

### Benzotriazole-1-carboxylic acid ethyl ester

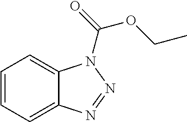

Ethyl chloroformate (0.46 mL, 4.8 mmol) was added to a stirred solution of 1H-benzotriazol (520 mg, 4.37 mmol) in dry THF (10 mL) and triethylamine (0.76 mL, 5.7 mmol) at ambient temperature. The reaction mixture was stirred overnight before the solvent was removed under reduced pressure and the residue purified by column chromatography on silica to give benzotriazole-1-carboxylic acid ethyl ester.

¹H-NMR (CDCl₃, 400 MHz) δ_{H} 8.05 (t, 2H, J 8.4 Hz), 7.59 (t, 1H, J 7.6 Hz), 7.43 (t, 1H, J 7.6 Hz), 4.62 (q, 2H, J 7.2 Hz), 1.52 (t, 3H, J 7.2 Hz).

¹³C-NMR (CDCl₃, 100 MHz) δ_{C} 148.9, 145.9, 131.8, 130.2, 125.7, 120.4, 113.5, 65.2, 14.3.

### Example 30

### 1-Naphthoic acid secbutyl ester

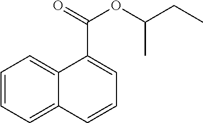

The title compound was prepared according to the procedure described in Example 1 from 1-napthoic acid and 2-bromobutane.

¹H-NMR (CDCl₃, 400 MHz) δ_{H} 8.91 (d, 1H, J 8.4 Hz), 8.16 (dd, 1H, J1.2 Hz, J 7.2 Hz), 8.01 (d, 1H, J 8.4 Hz), 7.88 (b d, 1H, J 8.0 Hz), 7.63-7.7.59 (m, 1H), 7.55-7.48 (m, 2H), 5.33 (sex, 1H, J 7.2 Hz), 1.88-1.68 (m, 2H), 1.42 (d, 3H, J 7.2 Hz), 1.04 (t, 3H, J 7.2 Hz). ¹³C-NMR (CDCl₃, 100 MHz) δ_{C} 167.6, 134.1, 133.2, 131.6, 130.0, 128.7, 128.3, 127.8, 126.4, 126.1, 124.7, 73.2, 29.3, 19.9, 10.1.

### Example 31

### 1-Naphthoic acid cyclopropylmethyl ester

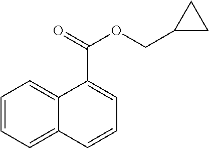

The title compound was prepared according to the procedure described in Example 1 from 1-napthoic acid and (bromomethyl)cyclopropane.

¹H-NMR (CDCl₃, 400 MHz) δ_{H} 8.91 (dd, 1H, J 1.2 Hz, J 8.8 Hz), 8.21 (dd, 1H, J 1.2 Hz, J 7.2 Hz), 8.20 (d, 1H, J 8.0 Hz), 7.88 (b d, 1H, J 8.0 Hz), 7.64-7.59 (m, 1H), 7.56-7.51 (m, 2H), 4.26 (d, 2H, J 7.2 Hz), 1.38-1.30 (m, 1H), 0.69-0.64 (m, 2H), 0.45-0.41 (m, 2H).

### Example 32

### 1-Naphthoic acid cyclopentyl ester

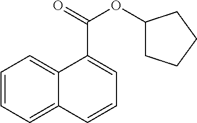

The title compound was prepared according to the procedure described in Example 1 from 1-napthoic acid and (bromomethyl)cyclopropane.

¹H-NMR (CDCl₃, 400 MHz) δ_{H} 8.89 (d, 1H, J 8.4 Hz), 8.13 (dd, 1H, J 1.2 Hz, J 7.2 Hz), 8.0 (d, 1H, J 8.4 Hz), 7.88 (d, 1H, J 8.4 Hz), 7.62-7.58 (m, 1H), 7.55-7.47 (m, 2H), 5.52 (tt, 1H, J 3.2 Hz, J 6.0 Hz), 2.08-1.66 (m, 8H).

### Example 33

### 1-Naphthoic acid cyclohexyl ester

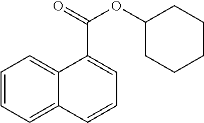

The title compound was prepared according to the procedure described in Example 1 from 1-napthoic acid and cyclohexyl bromide.

¹H-NMR (CDCl₃, 400 MHz) δ_{H} 8.90 (d, 1H, J 7.2 Hz), 8.17 (dd, 1H, J 2.0 Hz, J 7.2 Hz), 8.01 (d, 1H, J 8.0 Hz), 7.88 (dd, 1H, J 8.0 Hz), 7.63-7.59 (m, 1H), 7.55-7.48 (m, 2H), 5.15 (tt, 1H, J 4.0 Hz, 8.4 Hz), 2.07-2.03 (m, 2H), 1.85-1.81 (m, 2H), 1.71-1.35 (m, 6H).

¹³C-NMR (CDCl₃, 100 MHz) δ_{C} 167.4, 134.1, 133.2, 131.6, 130.1, 128.7, 128.3, 127.8, 126.4, 126.1, 124.8, 73.6, 32.0, 25.7, 24.1.

Spectral data was in accordance with: Ng, K. S., Roberts, J. L., Rutledge, P. S., Wilson, M. A., Woodgate, P. D. Aust. J. Chem. 1976, 29, 2683-2692.

### Example 34

### 2,3-Dihydro-indole-1-carboxylic acid ethyl ester

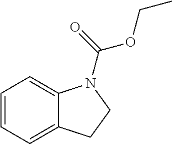

To a stirred solution of indoline (0.50 g, 4.2 mmol) in dry CH₂Cl₂ (25 mL) at 0° C. was added triethylamine (0.75 mL, 12.6 mmol) and ethyl chloroformate (0.82 mL, 6.3 mmol). The reaction mixture was stirred for 4 hours before it was diluted with CH₂Cl₂ and washed with hydrochlorid acid (0.1 M), dried over MgSO₄, filtered and concentrated. The material was purified by column chromatography on silica (petrol/AcOEt 9:1) to give the title compound as crystals.

¹H-NMR (CDCl₃, 400 MHz) δ_{H} 7.83 (b s, 2/3H), 7.45 (b s, 1/3H), 7.19-7.14 (m, 2H), 6.94 (t, 1H, J 7.2 Hz), 4.29 (b s, 2H), 4.01 (t, 2H, J 8.4 Hz), 3.11 (t, 2H, J 8.4 Hz), 1.36 (b s, 3H).

Spectral data was in accordance with: de Oliveira Baptista, M. J. V., Barrett, A. G. M., Barton, D. H. R., Girijavallabhan, M., Jennings, R. C., Kelly, J., Papadimitriou, V. J., Turner, J. V., Usher, N. A., J. Chem. Soc. Perkin Trans. 1. 1977, 1477-1500.

### Example 35

### 3-Methyl-indole-1-carboxylic acid ethyl ester

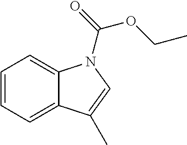

The title compound was prepared according to the procedure described in Example 24 from 3-methyl indole and ethyl chloroformate.

¹H-NMR (CDCl₃, 400 MHz) δ_{H} 8.15 (b d, J 6.4 Hz), 7.50 (dd, 1H, J 1.6 Hz, J 8.0 Hz), 7.39 (b s, 1H), 7.33 (dt, 1H, 0.8 Hz, 7.2 Hz), 7.28-7.24 (m, 2H), 4.47 (q, 2H, J 6.8 Hz), 2.28 (s, 3H), 1.46 (t, 3H, 6.8 Hz).

### Example 36

### (Naphthalen-1-ylsulfanyl)-acetonitrile

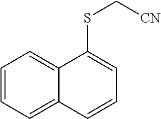

The title compound was prepared according to the procedure described in Example 11 from 1-napthalenethiol and chloroacetonitrile.

¹H-NMR (CDCl₃, 400 MHz) δ_{H} 8.44 (d, 1H, J 8.4 Hz), 7.95-7.91 (m, 3H), 7.65 (t, 1H, J 6.8 Hz), 7.58 (t, 1H, 6.8 Hz), 7.51 (t, 1H, J 7.2 Hz), 3.60 (s, 2H).

¹³C-NMR (CDCl₃, 100 MHz) δ_{C} 129.3, 128.9, 128.5, 125.7, 124.0, 123.7, 122.5, 121.6, 120.8, 119.7, 111.3, 16.2.

### Example 37

### 7-Methyl-indole-1-carboxylic acid ethyl ester

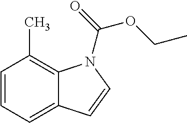

The title compound was prepared according to the procedure described in Example 24 from 7-methyl-indole and ethyl chloroformate.

¹H-NMR (CDCl₃, 400 MHz) δ_{H} 7.60 (d, 1H, J 4.0 Hz), 7.40 (b d, 1H, J 7.2 Hz), 7.19-7.39 (m, 2H), 6.57 (d, 1H, J 4.0 Hz), 4.45 (q, 2H, J 7.2 Hz), 2.66 (s, 3H), 1.45 (t, 3H, J 7.2 Hz).

¹³C-NMR (CDCl₃, 100 MHz) δ_{C} 151.2, 135.0, 132.2, 128.1, 128.0, 125.7, 123.6, 118.9, 108.3, 63.5, 22.5, 14.6.

Data was found to be in accordance with: A. K. Mohanakrishnan, R. Balamurugan, N. Ramesh, M. Mathiselvam, S. Manavalan, Synth. Comm. 2007, 37, 4343-4352.

### Example 38

### 4-Chloro-indole-1-carboxylic acid ethyl ester

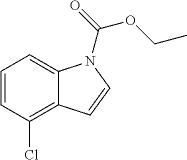

The title compound was prepared according to the procedure described in Example 24 from 4-chloro-indole and ethyl chloroformate.

¹H-NMR (CDCl₃, 400 MHz) δ_{H} 8.10 (t, 1H, J 4.4 Hz), 7.60 (d, 1H, J 3.6 Hz), 7.26-7.23 (m, 2H), 6.73 (dd, 1H, J 0.8 Hz, J 4.0 Hz), 4.51 (q, 2H, J 7.2 Hz), 1.48 (t, 3H, J 7.2 Hz).

### Example 39

### 7-Chloro-indole-1-carboxylic acid ethyl ester

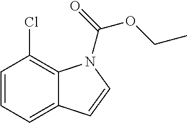

The title compound was prepared according to the procedure described in Example 24 from 7-chloro-indole and ethyl chloroformate.

¹H-NMR (CDCl₃, 400 MHz) δ_{H} 7.60 (d, 1H, J 3.6 Hz), 7.48 (dd, 1H, J 1.2 Hz, J 7.6 Hz), 7.34 (dd, 1H, J 1.2 Hz, 7.6 Hz), 7.18 (t, 1H, J 8.0 Hz), 6.60 (d. 1H, J 4.0 Hz), 4.49 (q, 2H, J 7.2 Hz), 1.45 (t, 3H, J 7.2 Hz).

¹³C-NMR (CDCl₃, 100 MHz) δ_{C} 150.5, 134.4, 132.3, 129.2, 127.0, 124.2, 120.8, 119.9, 107.8, 64.1, 14.5.

### Example 40

Dissociation Assay

Materials and Methods

Dulbecco's modified Eagle's medium, fetal bovine serum, trypsin, and penicillin/streptomycin were purchased from Invitrogen. Cell culture flasks were from NUNC. MicroScint-20 scintillation mixture was from Packard. [³H]-Citalopram (85 Ci/mmol) was a gift from H. Lundbeck A/S (Valby, Denmark). Fugene-6 transfection reagent was from Roche Molecular Biochemicals. All chemicals are commercially available. Stock solutions of the allosteric ligands were prepared as 100 mM in EtOH.

Transfection Protocols

Cell Culture and Expression of hSERT in HEK-293 MSR Cells.

HEK-293 MSR cells were maintained in Dulbecco's modified Eagle's medium (DMEM) supplemented with 10% fetal calf serum, 0.1 mM nonessential amino acids, 600 μg/mL Geneticin, 100 μg/ml streptomycin, and 100 units/ml penicillin at 37° C. and 5% CO₂ in a humidified atmosphere. The pcDNA3 plasmid vector containing the hSERT gene was used for transfection.

Membrane Preparations

17.5 μl Fugene-6 (Roche Molecular Biochemicals) were mixed with 280 μl Dulbecco's modified Eagle's medium, and incubated at room temperature for 5 minutes. 10 μg of hSERT plasmid were added and the mixture was further incubated at least 15 minutes. HEK-293 MSR cells were trypsinized and suspended in growth media, and the plasmid/Fugene-6 mixture was added. The cells were plated at 35% confluency in a 150 mm dish, and grown at 37° C. for 64 h. Prior to harvesting the dish was rinsed in PBS. Cells were harvested with a cell scraper in buffer 1 (50 mM Tris-base, 150 mM NaCl, 20 mM EDTA, pH 7.4). After centrifugation (3000 g at 4° C. for 10 mins), cells were suspended and homogenized with an IKA Ultra-Turrax from Rose Scientific Ltd (Edmonton, Alberta, Canada) for 20 s in buffer 1. Membranes were pelleted by ultracentrifugation (12 000 g at 4° C. for 15 min) and homogenization was repeated in buffer 1. Finally, after ultracentrifugation (12000 g, 4° C., 15 min) membranes were resuspended in 1 ml buffer 3 (50 mM Tris-base, 120 mM NaCl, 5 mM KCl, pH 7.4) and stored at −80° C.

Dissociation Assay with [³H]-Citalopram and Membrane Preparations.

SERT-[³H]-citalopram complex was formed by incubating hSERT membrane preparation and radioligand in buffer 3 during a 60 min incubation at 4° C. Radioligand was present at a concentration 10 times the K_{d} value. The time kinetic of dissociation was followed by adding 10 μL complex solution to 250 μL buffer 3 in 96-well plates and incubating subsequently for increasing time intervals at RT. Reactions were terminated by filtration through GF/C glass-fibre filters (Unifilter, Perkin Elmer Life Sciences), preincubated with 40 μl 0.5% polyethyleneimine, using a Pachard Bell cell harvester, and subsequently washed three times with water. Filters were soaked in 40 μl Microscint 20 scintillation liquid (Pachard Bell). Bound radioactivity was determined by direct counting of plates using a Packard Bell microplate scintillation counter. Dissociation curves were obtained by plotting residual binding vs. time of dissociation.

The allosteric ligands were tested for the ability to affect the dissociation of [³H]-Citalopram in the presence of 10 μM fluoxetine. Fluoxetine is devoid of allosteric potency against [³H]-Citalopram, and is included in the dissociation buffer in order to prevent reassociation of radioligand. The interaction of the allosteric ligand with the radioligand was studied by combining the allosteric ligand and fluoxetine in the dissociation buffer.

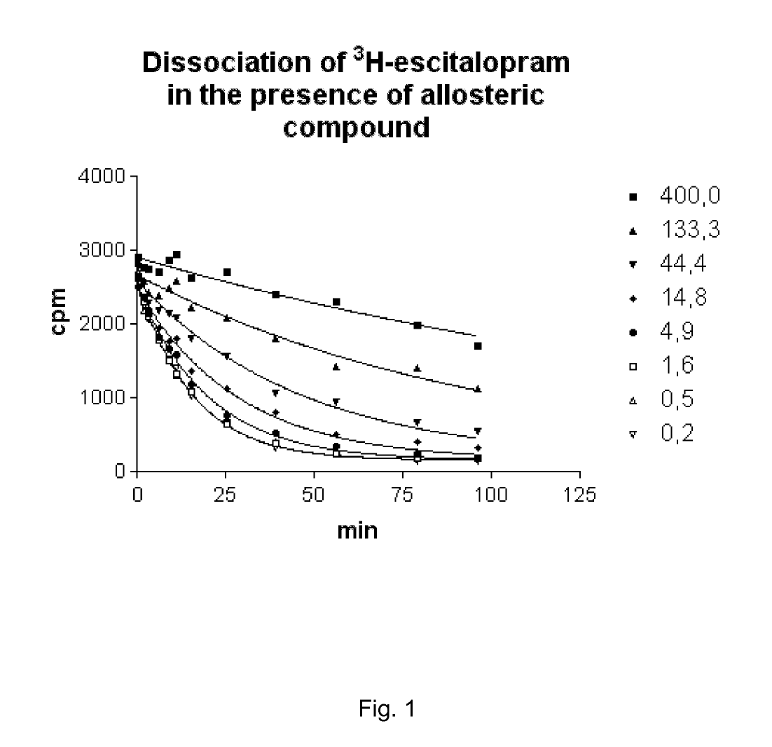

FIG. 1 gives the dissociation of [³H]-escitalopram from SERT in the presence of increasing amounts of the allosteric compound of Example 1. The concentrations of allosteric compound in the dissociation buffer range from 400-0.2 μM.

FIG. 2 shows the sigmoidal response of relative off-rate vs. concentration of allosteric compound. The relative off-rate is expressed as the off-rate of the radioligand at a given concentration of allosteric compound, normalized to the off-rate of radioligand in the absence of allosteric compound. The EC₅₀ value is determined as the concentration of allosteric compound which induces a 50% attenuation of the off-rate of the radioligand, compared to the off-rate of radioligand in the absence of allosteric compound. The EC₅₀ value for the compound of Example 1 is determined to be 3.3 μM.

FIG. 3 shows the curve used to determine the maximum stabilization factor for the compound of Example 1. The maximum stabilization factor is defined as the estimated upper plateau of the sigmoidal response curve, and describes how many folds the off-rate of bound radioligand from SERT can be attenuated in the presence of increasing concentrations of allosteric compound in the dissociation buffer. The maximum stabilization factor for the compound of Example 1 is determined to be factor 9.1.

Results

| Example | | EC₅₀ | Max. |
|---|---|---|---|
| No. | Structure | (μM) | Stability |
| 1 | 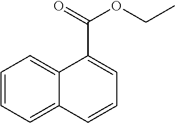 | 3.3 | 9.1 |
| 2 | 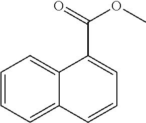 | 29 | 7.4 |
| 3 | 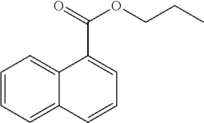 | 2.1 | 17 |
| 4 | 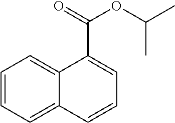 | 9.8 | 16 |
| 6 | 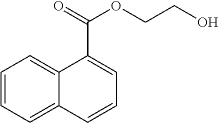 | 14.5 | 20 |
| 8 | 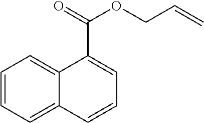 | 16 | 11 |
| 9 | 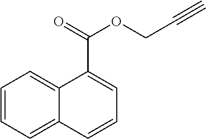 | 15 | 13 |
| 10 | 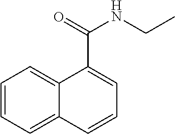 | 74 | 1.8 |
| 24 | 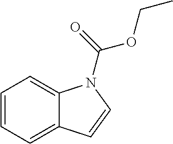 | 1.3 | 9 |
| 27 | 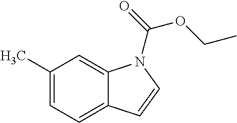 | 34 | 15 |
| 28 | 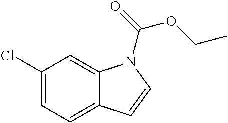 | 31 | 8.8 |
| 29 | 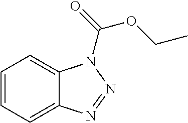 | 61 | 5.4 |
| 11 | 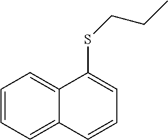 | 4.7 | 6.4 |
| 12 | 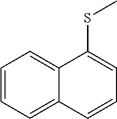 | 17 | 10 |
| 13 | 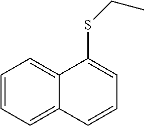 | 9.4 | 8.3 |
| 14 | 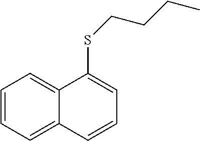 | 12 | 3.6 |
| 15 | 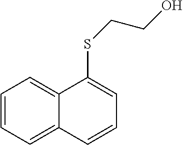 | 4.6 | 10.2 |
| 16 | 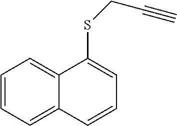 | 1.2 | 10.3 |
| 17 | 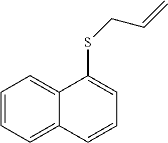 | 3.2 | 6.3 |
| 19 | 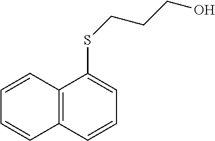 | 6.4 | 5.8 |
| 21 | 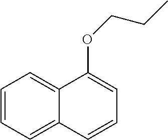 | 46 | 3.9 |
| 32 | 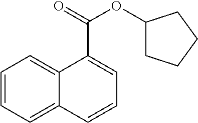 | 9.6 | 10 |
| 35 | 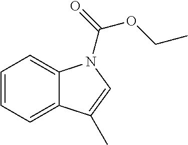 | 5 | 6 |
| 36 | 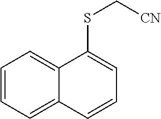 | 0.7 | 27 |
| Comparative compound not according to the invention | 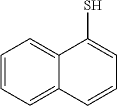 | >1000 | N/A |
| Comparative compound not according to the invention | 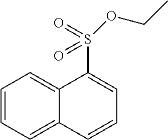 | 120 | 3.1 |

N/A = No activity

### Example 41

In Vitro Assay

Cell lines expressing the serotonin transporter constitute the most popular in vitro screening system for classical antidepressants, which will inhibit the transporter in uptake of serotonin. The inhibitory potency of the allosteric ligands is determined and expressed as an IC₅₀ value in that a lowering of the IC50 values, when the concentration of the allosteric ligands increases, indicates an add-on inhibitory effect due to the presence of the allosteric ligand.

Determination of Allo-Ligand Potentiating Effect on Uptake Inhibition by S-Citalopram.

Transfection of HEK-293-MSR Cell Cultures

For transfections, 0.2 μg of pcDNA3 plasmid vector containing a hSERT cDNA insert and 0.4 μl of Fugene6 (Roche) were used per cm2 of plating area. Plasmid and Fugene6 were mixed with DMEM according to the manufacturer's recommendations. HEK-293-MSR cells were treated with trypsin, suspended in growth media and added to the Plasmid/Fugene6 mixture. Transfected cells were dispensed into white 96-well growth plates (Corning) or 150 mm cell culture discs at 60-80% confluence.

5-HT Uptake Inhibition Assays

Uptake assays were performed 40-50 hours after transfection. For IC₅₀ determinations, transfected cells were washed with Phosphate Buffered Saline (PBS); 137 mM NaCl, 2.7 mM KCl, 4.3 mM Na₂HPO₄, 1.4 mM KH₂PO₄, supplemented with 0.1 mM CaCl₂ and 1 mM MgCl₂ (PBSCM) at pH 7.4 and immediately preincubated for 25 minutes at RT at 12 increasing concentrations of S-citalopram. Uptake was initiated by adding 30 μl allosteric ligand dissolved in PBSCM containing [³H]5-HT to a final 5-HT concentration of 70-100 nM. Incubation proceeded for 10 minutes before termination by aspiration and washing in PBSCM. The assay was terminated after 10 minutes by aspiration and washing in PBSCM. Cells were lysed in 50 μl MicroScint-20 (Packard), and the radiolabeled serotonin accumulated in the cells was quantified in a Packard TopCounter NXT microplate scintillation counter. Exact [³H]5-HT reaction concentrations were determined in a Packard TRI-CARB® Liquid scintillation analyzer. A series of IC₅₀ determinations were performed in the presence of a fixed concentration of allosteric ligand. The allosteric ligand concentrations were varied in the range from 0.1 μM to 5.0 μM.

FIG. 4 shows the results from the in vitro assay, in which the inhibitory effect of the allosteric compound (Naphthalen-1-ylsulfanyl)-acetonitrile (Example 36) was investigated. It is seen that the add-on effect of combining the allosteric compound with a conventional antidepressant such as S-citalopram reduces IC₅₀ values up to 2-fold when dosing the allosteric ligand in the 600 nM range.

### Example 42

Forced Swim Test

The forced swim test (FST) protocol is based on the on the work presented by Dulawa et al, Neuropsychopharmacology (2004) and Holick et al, Neuropsychopharmacolog (2008).

FST is a behavioral model widely used to assess antidepressant-like activity in mice Porsholt et al, 1978). The test is based on the fact that animals subjected to the short-term, inescapable stress of being placed in a water filled cylinder will perform swimming behavior interrupted by periods of immobility. The total duration of immobility within a seven minute test period, is the main parameter measured. Administration of antidepressants prior to FST will decrease the time spent in immobility. Thus, ligands assumed to potentiate antidepressants is predicted to cause a further decrease in immobility time when co-administered.

Balb/cJ male mice were used. Mice arrived at 6-8 weeks of age, and were tested between 12 and 18 weeks of age (25-30 g). For all experiments, mice were housed six per cage in a colony room controlled for light (12 h light/12 h dark; on from 0600 to 1800 h) and temperature. Food and water were available ad libitum. Forced swim testing occurred during the light phase between 1000 and 1600 h.

The allosteric ligands were dissolved in 10% beta-hydroxy-cyclodextrin, and incubated overnight at 4° C. on a tilting table. When combining the ligands with the SSRI fluoxetine, both compounds were dissolved in the vehicle solution and incubated overnight.

Male BalB/c were injected intraperitoneally with 500 μl SSRI alone or in combination with allosteric compound 30 min prior to the swimming session (n=6). Vehicle treated animals received 500 μl 10% β-2-hydroxy-cyclodextrine solution. Mice were placed in transparent plexiglass cylinder filled with 26° C. tap water for 7 min each. Swim sessions were videofilmed and analyzed by a blind scorer using stop watch, or by analyzing software (Ethovision, Noldus). The parameter measured was amount of time spent in a state of immobility, including small movements to keep balance and head over water. Immediately after the session the mice were briefly wiped with a paper towel and placed under a heating lamp for 15 min. Following this the mice were returned to the homecage.

FIGS. 5A and 5B gives the results from the forced swim test for the compound of Example 1. FIG. 5A depicts the effect of i) vehicle, ii) 5 mg/(kg bodyweight) of fluoxetine alone, and iii) 5 mg/(kg bodyweight) of fluoxetine in combination with 15 mg/(kg bodyweight) of the allosteric compound of Example 1, on the immobility in seconds. FIG. 5B depicts the effect of i) vehicle, ii) 5 mg/(kg bodyweight) of escitalopram alone, and iii) 5 mg/(kg bodyweight) of escitalopram in combination with 15 mg/(kg bodyweight) of the allosteric compound of Example 1. The dose of SSRI was chosen to be the highest sub-active concentration determined. The rationale behind this experimental design is that an SSRI-potentiating compound is expected to induce activity of a subactive dose of SSRI in the FST.

Accordingly, it can be seen from the FST results, that the allosteric ligand of Example 1 has a positive impact on the performance of the subactive doses of fluoxetine and escitalopram in FST. Accordingly, the combination of subactive escitalopram or fluoxetine with the compound of Example 1 induces a significantly reduced immobility time within a seven minutes scoring period.

### REFERENCES

Barker E L, Blakely R D. 1995. Norepinephrine and serotonin transporters. Molecular targets of antidepressant drugs. In: Bloom F E, Kupfer D F, editors. Psychopharmacology: the fourth generation of progress. Vol. 28. New York: Raven Press.

Chen F, Larsen M B, Neubauer H A, Sánchez C, Plenge P, Wiborg O. 2005. Characterization of an allosteric citalopram-binding site at the serotonin transporter. J Neurochem 92(1):21-8.

Dulawa S C, Holick K A, Gundersen B, Hen R. 2004. Effects of chronic fluoxetine in animal models of anxiety and depression. Neuropsychopharmacology. 29(7):1321-30.

Holick K A, Lee D C, Hen R, Dulawa S C. 2008. Behavioral effects of chronic fluoxetine in BALB/cJ mice do not require adult hippocampal neurogenesis or the serotonin 1A receptor. Neuropsychopharmacology. 33(2):406-17.

Neubauer H A, Hansen C G, Wiborg O. 2006. Dissection of an allosteric mechanism on the serotonin transporter: a cross-species study. Mol Pharmacol; 2006; 69(4); 1242-50.

Owens M J, Morgan W N, Plott S J, and Nemeroff C B. 1997. Neurotransmitter receptor and transporter binding profile of antidepressants and their metabolites. J Pharmacol Exp Ther 283:1305-1322.

Plenge P, Mellerup E T. 1985. Antidepressive drugs can change the affinity of [3H]imipramine and [3H]paroxetine binding to platelet and neuronal membranes. Eur J Pharmacol. 119(1-2); 1-8

Plenge P, Mellerup E T. 1997. An affinity-modulating site on neuronal monoamine transport proteins”; Pharmacol Toxicol. 80(4); 197-201

Plenge P, Mellerup E T, Laursen H. 1991. Affinity modulation of [3H]imipramine, [3H]paroxetine and [3H]citalopram binding to the 5-HT transporter from brain and platelets. Eur J Pharmacol. 206(3):243-50.

Porsolt R D, Le Pichon M, Jalfre M. 1977. Depression: a new animal model sensitive to antidepressant treatments. Nature. 266(5604):730-2.

Tatsumi M, Groshan K, Blakely R D, and Richelson E. 1997. Pharmacological profile of antidepressants and related compounds at human monoamine transporters. Eur J Pharmacol 340:249-258.

Wennogle L P, Meyerson L R. 1982. Serotonin modulates the dissociation of [3H]imipramine from human platelet recognition sites. Eur J Pharmacol. 86(2); 303-7

Wennogle L P, Meyerson L R. 1985. Serotonin uptake inhibitors differentially modulate high affinity imipramine dissociation in human platelet membranes. Life Sci. 36(16); 1541-50.)

1. A method of treating a central nervous system (CNS) disorder in a subject in need of such treatment, comprising administering to said subject a therapeutically effective amount of a compound of formula (I) 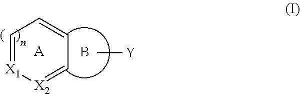
or a pharmaceutical acceptable salt, solvate or prodrug thereof;
wherein
Y is selected from 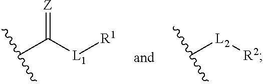
A is a 5- or 6-membered aryl or heteroaryl ring;
n is 0 or 1;
B is a 4-, 5-, or 6-membered cycloalkyl, aryl, heterocyclyl or heteroaryl ring, which ring together with A forms an annulated ring system;
X₁ and X₂ are each independently an atom selected from the group consisting of Carbon, Nitrogen, Oxygen, and Sulphur, wherein at least one of X₁ and X₂ is Carbon;
Z is an atom selected from the group consisting of Oxygen and Sulphur, with the proviso that when Z is Sulphur, then L₁ is —NH— or —NR³—;
L₁ is a linker selected from the group consisting of —O—, —NH—, —NR³—, and —C—;
L₂ is a linker selected from the group consisting of —O—, —S—, —NH—, and —NR⁴—;
R¹ is selected from the group consisting of C₁₋₈ alkyl, C₁₋₈ alkoxy, C₂₋₈ alkenyl, C₂₋₈ alkynyl, C₃₋₁₀ cycloalkyl, aryl, heterocyclyl, heteroaryl, —NH—C₁₋₆ alkyl, C₃₋₁₀ cycloalkyl-C₁₋₆ alkyl, aryl-C₁₋₆ alkyl, heterocyclyl-C₁₋₆ alkyl, and heteroaryl-C₁₋₆ alkyl;
R² is selected from the group consisting of C₁₋₈ alkyl, C₂₋₈ alkenyl, C₂₋₈ alkynyl, C₃₋₁₀ cycloalkyl, aryl, heterocyclyl, heteroaryl, C₃₋₁₀ cycloalkyl-C₁₋₆ alkyl, aryl-C₁₋₆ alkyl, heterocyclyl-C₁₋₆ alkyl, and heteroaryl-C₁₋₆ alkyl;
where any alkyl, alkenyl, alkynyl, cycloalkyl, aryl, heterocyclyl, and heteroaryl of R¹ and R² each independently optionally is substituted with one or more substituents selected from the group consisting of halogen, —OH, —SH, —NO₂, —CN, —NH₂, —N₃, C₁₋₆ alkyl, C₁₋₆ alkoxy, —COOH, —C(O)O—(C₁₋₆ alkyl), —C(O)—NH₂, —C(O)—NH(C₁₋₄ alkyl), —NH(C₁₋₆ alkyl), —N(C₁₋₄ alkyl)(C₁₋₄ alkyl), —NHC(O)—(C₁₋₆ alkyl), —S—(C₁₋₄ alkyl), —S(O)—(C₁₋₄ alkyl), —SO₂—(C₁₋₄ alkyl), —CCl₃, —CF₃, and —CH₂CF₃;
R³ is selected from the group consisting of C₁₋₆ alkyl, C₂₋₆ alkenyl, and C₂₋₆ alkynyl;
R⁴ is selected from the group consisting of C₁₋₆ alkyl, C₂₋₆ alkenyl, and C₂₋₆ alkynyl; and
where ring A and ring B of formula (I) each independently optionally is substituted with one or more substituents selected from the group consisting of halogen, —OH, —SH, —NO₂, —CN, —NH₂, —N₃, C₁₋₄ alkyl, C₁₋₄ alkoxy, C₂₋₄ alkenyl, C₂₋₄ alkynyl, —COOH, —C(O)—NH₂, —NH(C₁₋₄ alkyl), —S—(C₁₋₄ alkyl), —CF₃, and —CH₂CF₃.

2. The method according to claim 1, comprising administering a compound of formula (I) wherein A is an aryl ring.

3-5. (canceled)

6. The method according to claim 1, comprising administering a compound of formula (I), wherein B is a 6-membered cycloalkyl, aryl, or heteroaryl ring, which ring together with A forms an annulated ring system.

7-8. (canceled)

9. The method according to claim 1, comprising administering a compound of formula (I), wherein B is a 5-membered heteroaryl ring, which ring together with A forms an annulated ring system.

10-11. (canceled)

12. The method according to claim 1, comprising administering a compound for formula (I), wherein Y is

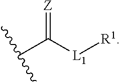

13. The method according to claim 12, wherein Z is an Oxygen atom, and Y then is

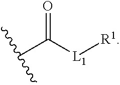

14. (canceled)

15. The method according to claim 1, comprising administering a compound of formula (I), wherein L₁ is selected from the group consisting of —O—, —NH—, and —NR³—.

16. The method according to claim 15, wherein R³ is C₁₋₄ alkyl.

17. The method according to claim 16, wherein R³ is selected from the group consisting of methyl, ethyl, propyl, isopropyolo, butyl, iso-butyl, sec-butyl, and tert-butyl.

18-21. (canceled)

22. The method according to claim 1, comprising administering a compound of formula (I), wherein R¹ is selected from the group consisting of C₁₋₆ alkyl, C₁₋₆ alkoxy, C₂₋₆ alkenyl, C₂₋₆ alkynyl, and —NH—C₁₋₆ alkyl, where any of these optionally is substituted with one or more substituents.

23. (canceled)

24. The method according to claim 22, wherein R¹ is C₁₋₄ alkyl, wherein the alkyl optionally is substituted with one or more substituents.

25-27. (canceled)

28. The method according to claim 1, comprising administering a compound of formula (I), wherein Y is

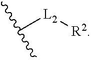

29. The method according to claim 28, wherein L₂ is selected from the group consisting of —O— and —S—.

30-38. (canceled)

39. The method according to claim 1, comprising administering a compound of formula (I), wherein R² is selected from the group consisting of C₁₋₄ alkyl, C₂₋₄ alkenyl, C₂₋₄ alkynyl, and C₃₋₅ cycloalkyl, where any of these optionally is substituted with one or more substituents.

40. (canceled)

41. The method according to claim 39, wherein R² is C₁₋₄ alkyl, wherein the alkyl optionally is substituted with one or more substituents.

42-45. (canceled)

46. The method according to claim 1, comprising administering a compound of formula (I), wherein any alkyl, alkenyl, alkynyl, cycloalkyl, aryl, heterocyclyl, and heteroaryl of R¹ and R² each independently optionally is substituted with one or two substituents selected from the group consisting of —OH, —CN, —N₃, —CCl₃, —CF₃, and —C(O)O—(C₁₋₂ alkyl).

47. The method according to claim 1, comprising administering a compound of formula (I), wherein ring A and ring B each independently optionally is substituted with one or more substituents selected from the group consisting of halogen, —OH, —CN, —N₃, C₁₋₄ alkyl, C₁₋₄ alkoxy, and —CF₃.

48-49. (canceled)

50. The method according to claim 1, comprising administering a compound of formula (I), wherein said compounds of formula (I) is of formula (Ia) 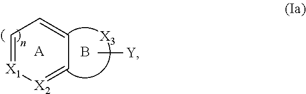
or a pharmaceutically acceptable salt, solvate or prodrug thereof,
wherein X₃ is an atom selected from the group consisting of Carbon, Nitrogen, Oxygen, and Sulphur; and wherein
A, B, X₁, X₂, Y, n, Z, L₁, L₂, R¹, R², R³, and R⁴ are as defined in claim 1.

51. The method according to claim 50, wherein X₃ is an atom selected from carbon and nitrogen.

52-55. (canceled)

56. The method according to claim 1, comprising administering a compound for formula (I), wherein compounds of formula (I) is of formula (IV) 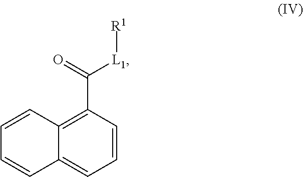
or a pharmaceutically acceptable salt, solvate or prodrug thereof,
wherein L₁, R¹, and R³ are as defined in claim 1.

57. The method according to claim 1, comprising administering a compound for formula (I), wherein compounds of formula (I) is of formula (VI) 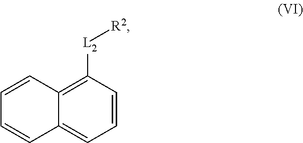
or a pharmaceutically acceptable salt, solvate or prodrug thereof,
wherein L₂, R², and R⁴ are as defined in claim 1.

58. The method according to claim 1, comprising administering a compound for formula (I), wherein compounds of formula (I) is of formula (V) 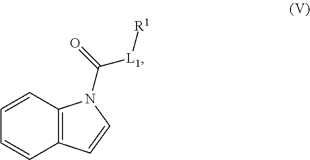
or a pharmaceutically acceptable salt, solvate or prodrug thereof,
wherein L₁, R¹, and R³ are as defined in claim 1.

59. The method according to claim 1, comprising administering a compound of formula I, wherein the compound is selected from
1-Naphthoic acid methyl ester;
1-Naphthoic acid ethyl ester;
1-Naphthoic acid isopropyl ester;
1-Naphthoic acid propyl ester;
1-Naphthoic acid 2-hydroxyethyl ester;
1-Naphthoic acid ethylamide;
1-Naphthoic acid pentyl ester;
1-Naphthoic acid 2-propenyl ester;
1-Naphthoic acid 2-propynyl ester;
1-Naphthoic acid secbutyl ester;
1-Naphthoic acid cyclopropylmethyl ester;
1-Naphthoic acid cyclopentyl ester;
1-Naphthoic acid cyclohexyl ester;
1-Naphthoic acid-2-methoxyethyl ester;
1-Naphthoic acid-2-methylsulfanyl ester;
(±) 1,2,3,4-Tetrahydro-1-naphthoic acid ethyl ester;
Benzotriazole-1-carboxylic acid ethyl ester;
2,3-Dihydro-indole-1-carboxylic acid ethyl ester;
Indole-1-carboxylic acid ethyl ester;
4-Methyl-indole-1-carboxylic acid ethyl ester;
5-Methyl-indole-1-carboxylic acid ethyl ester;
6-Methyl-indole-1-carboxylic acid ethyl ester;
6-Chloro-indole-1-carboxylic acid ethyl ester;
3-Methyl-indole-1-carboxylic acid ethyl ester;
7-Methyl-indole-1-carboxylic acid ethyl ester;
4-Chloro-indole-1-carboxylic acid ethyl ester;
7-Chloro-indole-1-carboxylic acid ethyl ester;
Indole-1-carboxylic acid 1,1,1-trichloroethyl ester;
7-Hydroxy-indole-1-carboxylic acid ethyl ester;
6-Hydroxy-indole-1-carboxylic acid ethyl ester;
5-Hydroxy-indole-1-carboxylic acid ethyl ester;
4-Hydroxy-indole-1-carboxylic acid ethyl ester;
7-Methoxy-indole-1-carboxylic acid ethyl ester;
6-Methoxy-indole-1-carboxylic acid ethyl ester;
5-Methoxy-indole-1-carboxylic acid ethyl ester;
4-Methoxy-indole-1-carboxylic acid ethyl ester;
5-Chloro-indole-1-carboxylic acid ethyl ester;
Indole-1-carboxylic acid ethyl amide;
Indole-1-carbothioic acid ethyl amide;
1-Propoxy-naphthalene;
Methyl-[1]naphthyl sulphide;
Ethyl-[1]naphthyl sulphide;
1-Propyl-sulfanyl-1-naphthalene;
1-Butyl-sulfanyl-1-naphthalene;
3-Ethenyl-sulfanyl-1-naphthalene;
3-Ethynyl-sulfanyl-1-naphthalene;
2-Hydroxyethyl-1-naphthyl sulphide;
3-Hydroxypropyl-1-naphthyl sulphide;
3-Dimethylaminopropyl-1-naphthyl sulphide;
Methyl β-(1-naphthylthio)-propionate;
β-(1-Naphthylthio)-propionic acid;
(Naphthalen-1-ylsulfanyl)-acetonitrile;
2-Dimethylaminoethyl-1-naphthyl sulphide;
Isopropyl-1-naphthyl sulphide;
sec-Butyl-1-naphthyl sulfide;
isobutyl-1-naphthyl sulfide;
Cyclohexyl-1-naphthyl sulphide;
Cyclopentyl-1-naphthyl sulphide;
(2-Methoxy-ethylsulfanyl)-1-naphthalene;
7-Ethylsulfanyl-1H-indole;
7-Ethylsulfanyl-benzofuran;
4-Ethylsulfanyl-1H-indole; and
4-Ethylsulfanyl-benzofuran.

60. The method of claim 1, wherein the compound is selected from
1-Naphthoic acid methyl ester;
1-Naphthoic acid ethyl ester;
1-Naphthoic acid isopropyl ester;
1-Naphthoic acid propyl ester;
1-Naphthoic acid 2-hydroxyethyl ester;
1-Naphthoic acid ethylamide;
1-Naphthoic acid pentyl ester;
1-Naphthoic acid 2-propenyl ester;
1-Naphthoic acid 2-propynyl ester;
1-Naphthoic acid secbutyl ester;
1-Naphthoic acid cyclopropylmethyl ester;
1-Naphthoic acid cyclopentyl ester;
1-Naphthoic acid cyclohexyl ester;
(±) 1,2,3,4-Tetrahydro-1-naphthoic acid ethyl ester;
Benzotriazole-1-carboxylic acid ethyl ester;
2,3-Dihydro-indole-1-carboxylic acid ethyl ester;
Indole-1-carboxylic acid ethyl ester;
4-Methyl-indole-1-carboxylic acid ethyl ester;
5-Methyl-indole-1-carboxylic acid ethyl ester;
6-Methyl-indole-1-carboxylic acid ethyl ester;
6-Chloro-indole-1-carboxylic acid ethyl ester;
3-Methyl-indole-1-carboxylic acid ethyl ester;
7-Methyl-indole-1-carboxylic acid ethyl ester;
4-Chloro-indole-1-carboxylic acid ethyl ester;
7-Chloro-indole-1-carboxylic acid ethyl ester;
1-Propoxy-naphthalene;
Methyl-[1]naphthyl sulphide;
Ethyl-[1]naphthyl sulphide;
1-Propyl-sulfanyl-1-naphthalene;
1-Butyl-sulfanyl-1-naphthalene;
3-Ethenyl-sulfanyl-1-naphthalene;
3-Ethynyl-sulfanyl-1-naphthalene;
2-Hydroxyethyl-1-naphthyl sulphide;
3-Hydroxypropyl-1-naphthyl sulphide;
3-Dimethylaminopropyl-1-naphthyl sulphide;
Methyl β-(1-naphthylthio)-propionate; and
β-(1-Naphthylthio)-propionic acid.

61. The method according to claim 1, wherein the CNS disorder is selected from the group consisting of depression, panic disorder, anxiety, obsessive-compulsive disorder (OCD), generalized anxiety disorder (GAD), social phobia, bulimia nervosa, anorexia nervosa, post-traumatic stress disorder (PTSD), and neuropathic pain.

62. The method according to claim 1, wherein the CNS disorder is selected from the group consisting of depression, panic disorder, anxiety, and obsessive-compulsive disorder (OCD) and wherein the CNS disorder is depression.

63. (canceled)

64. The method according to claim 1, wherein the compound is administered in combination with one or more further active substances, wherein the one or more further active substances are one or more psychiatric medications, or are one or more antidepressants.

65-66. (canceled)

67. The method according to claim 64, wherein the one or more further active substances are selected from the group consisting of selective serotonin reuptake inhibitors (SSRIs), tricyclic antidepressiva (TCAs), serotonin-norepinephrine reuptake inhibitors (SNRIs), and Serotonin-norepinephrine-dopamine-reuptake-inhibitors (SNDRIs).

68. The method according to claim 64, wherein the one or more further active substances are selective serotonin reuptake inhibitors (SSRIs) selected from the group consisting of citalopram, escitalopram, fluoxetine, paroxetine, sertraline, fluvoxamine, venlafaxine, duloxetine, zimelidine, and dapoxetine.

69-72. (canceled)

73. A pharmaceutical composition comprising a compound as defined in claim 1, and optionally one or more pharmaceutically acceptable excipients, diluents or carriers; said pharmaceutical composition may optionally comprise one or more further active substances; wherein the one or more further active substances are one or more psychiatric medications; wherein the one or more further active substances are antidepressants.

74. The pharmaceutical composition according to claim 73, wherein the one or more further active substances are as defined in claim 67.

75. The pharmaceutical composition according to claim 73, wherein the one or more further active substances are as defined in claim 68.

76-78. (canceled)

79. A compound of formula (I) 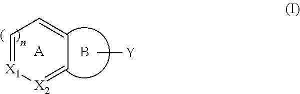
or a pharmaceutical acceptable salt, solvate or prodrug thereof;
wherein
Y is selected from 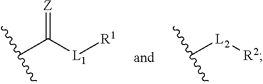
A is a 5- or 6-membered aryl or heteroaryl ring;
n is 0 or 1;
B is a 4-, 5-, or 6-membered cycloalkyl, aryl, heterocyclyl or heteroaryl ring, which ring together with A forms an annulated ring system;
X₁ and X₂ are each independently an atom selected from the group consisting of Carbon, Nitrogen, Oxygen, and Sulphur, wherein at least one of X₁ and X₂ is Carbon;
Z is an atom selected from the group consisting of Oxygen and Sulphur, with the proviso that when Z is Sulphur, then L₁ is —NH— or —NR³—;
L₁ is a linker selected from the group consisting of —O—, —NH—, —NR³—, and —C—;
L₂ is a linker selected from the group consisting of —O—, —S—, —NH—, and —NR⁴—;
R¹ is selected from the group consisting of C₁₋₈ alkyl, C₁₋₈ alkoxy, C₂₋₈ alkenyl, C₂₋₈ alkynyl, C₃₋₁₀ cycloalkyl, aryl, heterocyclyl, heteroaryl, —NH—C₁₋₆ alkyl, C₃₋₁₀ cycloalkyl-C₁₋₆ alkyl, aryl-C₁₋₆ alkyl, heterocyclyl-C₁₋₆ alkyl, and heteroaryl-C₁₋₆ alkyl;
R² is selected from the group consisting of C₁₋₈ alkyl, C₂₋₈ alkenyl, C₂₋₈ alkynyl, C₃₋₁₀ cycloalkyl, aryl, heterocyclyl, heteroaryl, C₃₋₁₀ cycloalkyl-C₁₋₆ alkyl, aryl-C₁₋₆ alkyl, heterocyclyl-C₁₋₆ alkyl, and heteroaryl-C₁₋₆ alkyl;
where any alkyl, alkenyl, alkynyl, cycloalkyl, aryl, heterocyclyl, and heteroaryl of R¹ and R² each independently optionally is substituted with one or more substituents selected from the group consisting of halogen, —OH, —SH, —NO₂, —CN, —NH₂—N₃, C₁₋₆ alkyl, C₁₋₆ alkoxy, —COOH, —C(O)O—(C₁₋₆ alkyl), —C(O)—NH₂, —C(O)—NH(C₁₋₄ alkyl), —NH(C₁₋₆ alkyl), —N(C₁₋₄ alkyl)(C₁₋₄ alkyl), —NHC(O)—(C₁₋₆ alkyl), —S—(C₁₋₄ alkyl), —S(O)—(C₁₋₄ alkyl), —SO₂—(C₁₋₄ alkyl), —CCl₃, —CF₃, and —CH₂CF₃;
R³ is selected from the group consisting of C₁₋₆ alkyl, C₂₋₆ alkenyl, and C₂₋₆ alkynyl;
R⁴ is selected from the group consisting of C₁₋₆ alkyl, C₂₋₆ alkenyl, and C₂₋₆ alkynyl; and
where ring A and ring B of formula (I) each independently optionally is substituted with one or more substituents selected from the group consisting of halogen, —OH, —SH, —NO₂, —CN, —NH₂, —N₃, C₁₋₄ alkyl, C₁₋₄ alkoxy, C₂₋₄ alkenyl, C₂₋₄ alkynyl, —COOH, —C(O)—NH₂, —NH(C₁₋₄ alkyl), —S—(C₁₋₄ alkyl), —CF₃, and —CH₂CF₃.
